(19) United States
(12) Patent Application Publication
(10) Pub. No.: US 2016/0039852 A1
(43) Pub. Date: Feb. 11, 2016

(54) THERAPEUTIC USES OF BISPHOSPHONATES

(71) Applicant: THE UNIVERSITY OF SHEFFIELD, Sheffield, South Yorkshire (GB)

(72) Inventors: Robert Graham-Goodwin Russell, Sheffield (GB); Ilaria Bellantuono, Sheffield (GB)

(21) Appl. No.: 14/781,661

(22) PCT Filed: Apr. 1, 2014
(86) PCT No.: PCT/GB2014/051019

§ 371 Date: Oct. 1, 2015

(30) Foreign Application Priority Data
Apr. 2, 2013 (GB) 1305947.2

(51) Int. Cl. C07F 9/6506; A61Q 17/04; A61K 8/55; A61K 45/06; C07F 9/6561; A61K 31/675

(52) U.S. Cl. CPC C07F 9/6506; C07F 9/6561; A61K 31/675; A61K 8/55; A61K 45/06; A61Q 17/04

(57) ABSTRACT

The invention relates to a bisphosphonate (BP) compound, or a pharmaceutically acceptable salt or solvate or prodrug thereof, for use as a cytoprotectant for protecting non-cancerous cells of a subject against radiation-induced damage and/or damage induced by a chemical agent.

### FIELD OF THE INVENTION

The invention relates to new uses of bisphosphonate (BP) compounds as cytoprotectants for promoting cell survival in vitro and in vivo. The invention provides new means for protecting cells against damage (for example, DNA damage), and in particular, damage caused by radiation and/or chemical agents.

### BACKGROUND TO THE INVENTION

Cells and tissues, and in particular, cellular DNA, are vulnerable to damage.

Damage may arise from exposure to radiation or chemical agents, both exogeneous and endogeneous. Such agents may, for example, occur in the environment. For example, cells may be damaged by exposure to solar radiation. Cells may also be exposed to damaging agents during medical or other treatments. For example, radiotherapy or chemotherapy, administered to kill cancerous cells in a subject, may cause damage to healthy non-target cells which are also exposed during treatment. Endogeneous chemical agents include reactive oxygen species, generated by natural metabolic processes in a cell.

Cells generally possess one or more repair mechanisms to restore damage, including DNA damage. Damage which is unrepaired, for example, because of an increased rate of damage, and/or a defective repair mechanism, can accumulate in cells. Accumulation of damage generally has undesirable consequences. For example, unrepaired DNA damage can cause an increased propensity to develop a primary cancer, or cell death. Accumulated damage in stem cells can lead to a reduced capacity to regenerate tissue, either for tissue maintenance in the life cycle of the organism, or for tissue repair, in response to tissue damage by disease or injury

### SUMMARY OF THE INVENTION

The present inventors have surprisingly found that bisphosphonate (BP) compounds may be used to protect cells against damage, for example DNA damage. Damage may be that induced by radiation and/or one or more chemical agents.

Accordingly, in one aspect, the invention provides a bisphosphonate (BP) compound, or a pharmaceutically acceptable salt or solvate or pro-drug thereof, for use as a cytoprotectant for protecting non-cancerous cells of a subject against radiation-induced damage and/or damage induced by a chemical agent.

The invention further provides:
a bisphosphonate (BP) compound, or a pharmaceutically acceptable salt or solvate or pro-drug thereof, for use as a cytoprotective adjuvant in cancer chemotherapy and/or cancer radiotherapy in a subject;
a bisphosphonate (BP) compound, or a pharmaceutically acceptable salt or solvate or pro-drug thereof, for use in a subject as a cytoprotectant for protecting non-cancerous cells against radiation-induced damage and/or damage induced by a chemical agent
a bisphosphonate (BP) compound, or a pharmaceutically acceptable salt or solvate or pro-drug thereof, for use in protecting a subject against damage by solar radiation;
use of a bisphosphonate (BP) compound, or a pharmaceutically acceptable salt or solvate or pro-drug thereof, for the manufacture of a cytoprotectant medicament for protecting non-cancerous cells of a subject against radiation-induced damage and/or damage induced by a chemical agent;
a method of protecting non-cancerous cells against radiation-induced damage and/or damage induced by a chemical agent, the method comprising administering an effective amount of a bisphosphonate (BP) compound, or a pharmaceutically acceptable salt or solvate or pro-drug thereof, to the cells;
use of a bisphosphonate (BP) compound, or a pharmaceutically acceptable salt or solvate or pro-drug thereof as a cytoprotectant for protecting non-cancerous cells against radiation-induced damage and/or damage induced by a chemical agent;
use of a bisphosphonate (BP) compound, or pharmaceutically acceptable salt or solvate or pro-drug thereof for preparing induced pluripotent stem cells;
a method of preparing induced pluripotent stem cells, the method comprising:
   administering an effective amount of a bisphosphonate (BP) compound, or a pharmaceutically acceptable salt or solvate or pro-drug thereof, to one or more multipotent cells; and
   preparing induced pluripotent stem cells from the multipotent cells;
use of a bisphosphonate (BP) compound, or pharmaceutically acceptable salt or solvate or pro-drug thereof as a cytoprotectant for treating cosmetic signs of aging in a subject or for protecting a subject against cosmetic damage by solar radiation;
a bisphosphonate (BP) compound, or a pharmaceutically acceptable salt or solvate thereof or pro-drug for use as a UV protectant;
use of a bisphosphonate (BP) compound, or a pharmaceutically acceptable salt or solvate or pro-drug thereof for reducing one or more visible signs of aging in skin;
a combination product for use in protecting non-cancerous cells of a subject against radiation-induced damage and/or damage induced by a chemical agent, the combination product comprising a bisphosphonate (BP) compound or a pharmaceutically acceptable salt or solvate or pro-drug thereof, and one or more agents selected from: cancer radiotherapy; cancer chemotherapeutic agents; cytoprotective agents; inhibitors of the mevalonate pathway; inhibitors of mTOR signalling; anti-inflammatory agents; immunomodulatory agents; UV-protectants;anti-infectives, and cardiac medications for heart disease and cardiovascular conditions;
a cytoprotective adjuvant composition comprising a bisphosphonate (BP) compound, or a pharmaceutically acceptable salt or solvate or pro-drug thereof, and a suitable carrier, excipient or diluent;
a UV protectant composition or sunscreen composition comprising a bisphosphonate (BP) compound, or a pharmaceutically acceptable salt or solvate or pro-drug thereof, and a suitable carrier, excipient or diluent;
a skincare composition, comprising a bisphosphonate (BP) compound, or a pharmaceutically acceptable salt or solvate or pro-drug thereof, and a suitable carrier, excipient or diluent.
cell growth media composition, or an additive composition for cell culture media comprising a bisphosphonate (BP) compound, or a pharmaceutically acceptable salt or solvate or pro-drug thereof, and a suitable carrier, excipient or diluent;
a bisphosphonate (BP) compound or a pharmaceutically acceptable salt or solvate or pro-drug thereof for use in protecting a subject against radiation-induced damage and/or damage induced by a chemical agent;
a bisphosphonate (BP) compound or a pharmaceutically acceptable salt or solvate or pro-drug thereof for use in reducing one or more side effect of radiotherapy and/or chemotherapy in a subject;
a compound selected from:
   (a) a phosphono-phosphinate compound; and
   (b) an inhibitor of FPPS enzyme;
   or a pharmaceutically acceptable salt or solvate or pro-drug of (a) or (b), for use as a cytoprotectant for protecting non-cancerous cells of a subject against radiation-induced damage and/or damage induced by a chemical agent;
a method of protecting non-cancerous cells against radiation-induced damage and/or damage induced by a chemical agent, the method comprising administering an effective amount of
   (a) a phosphono-phosphinate compound; or
   (b) an inhibitor of FPPS enzyme; or
   a pharmaceutically acceptable salt or solvate or pro-drug of (a) or (b) to the cells.

### DESCRIPTION OF THE FIGURES

FIG. 1: Human mesenchymal stem cells (hMSC) cultured in the presence of Zoledronate (Zol) showed extension of life span.

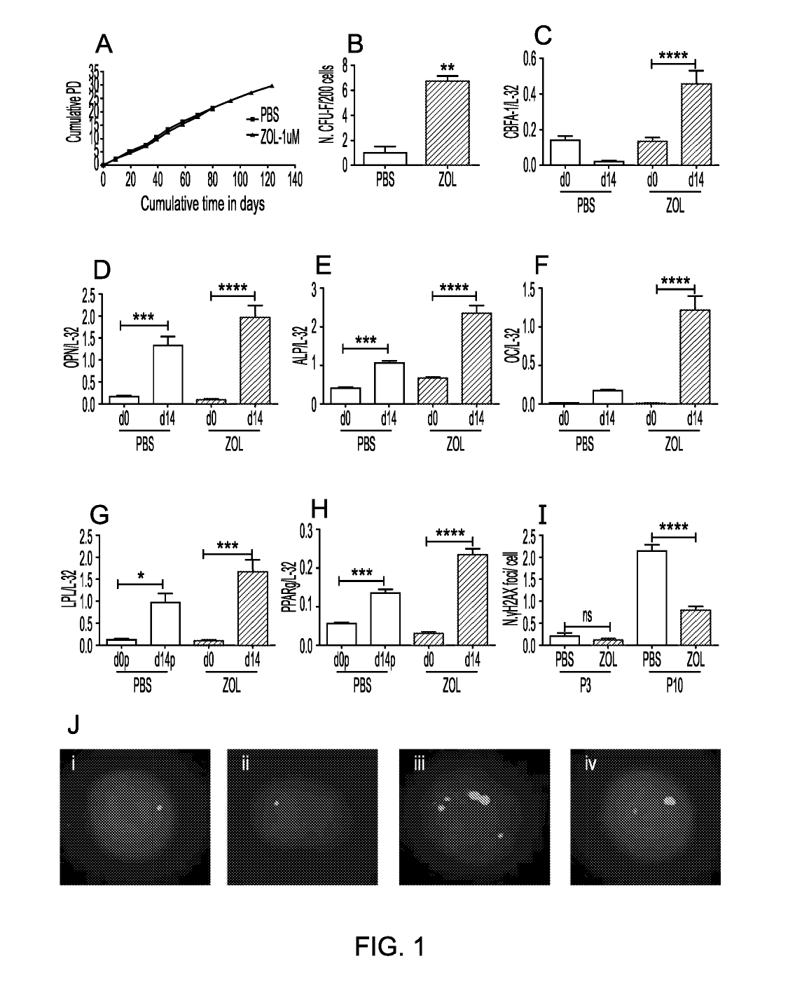

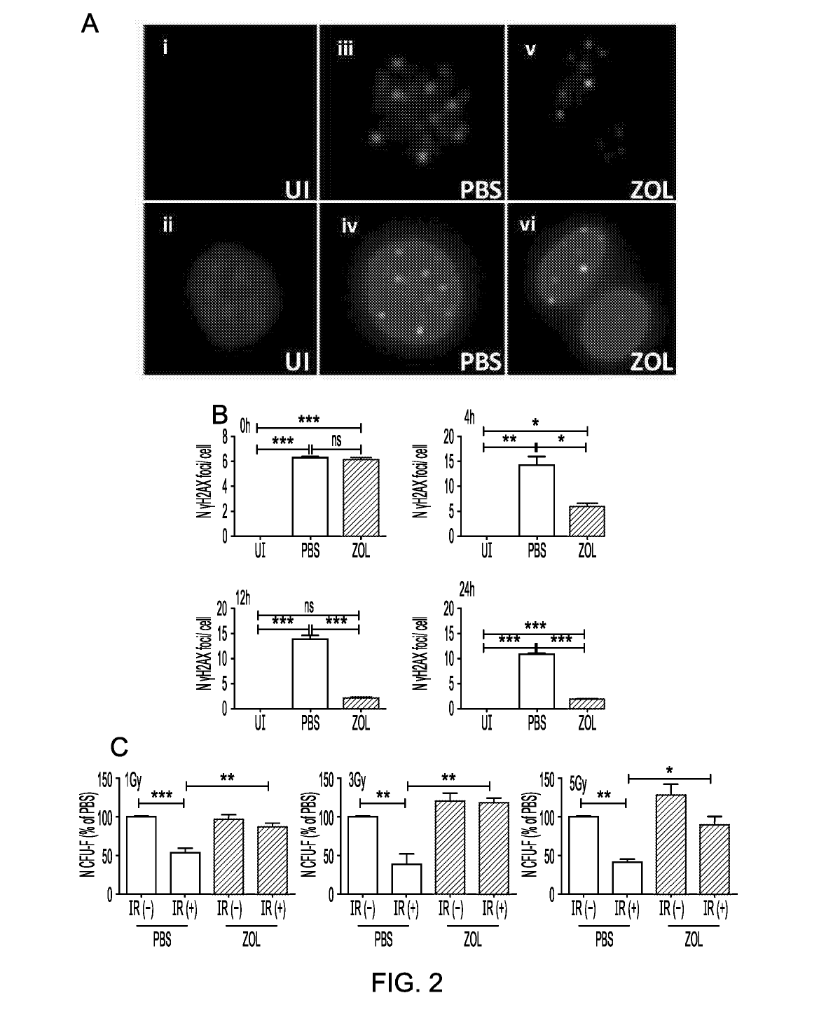

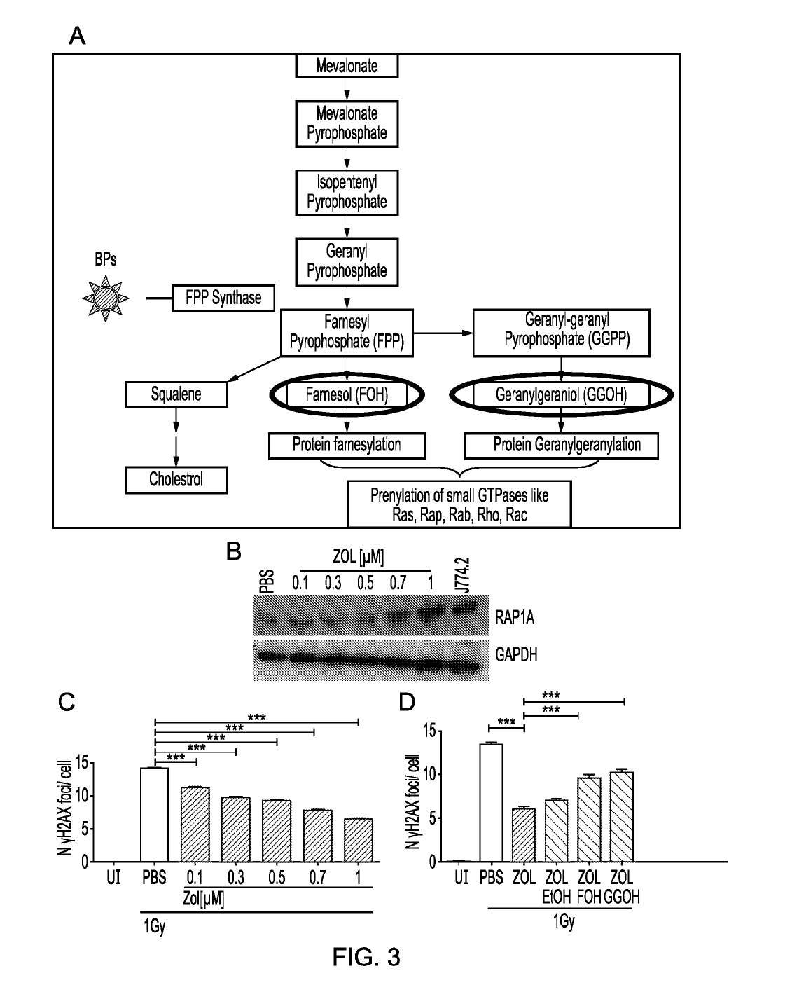

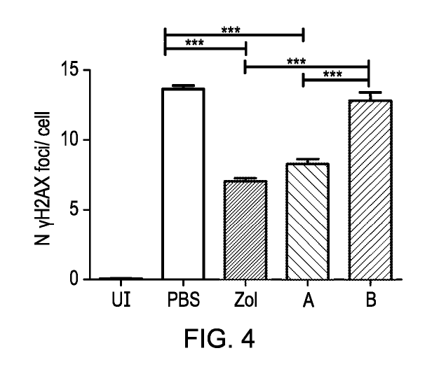

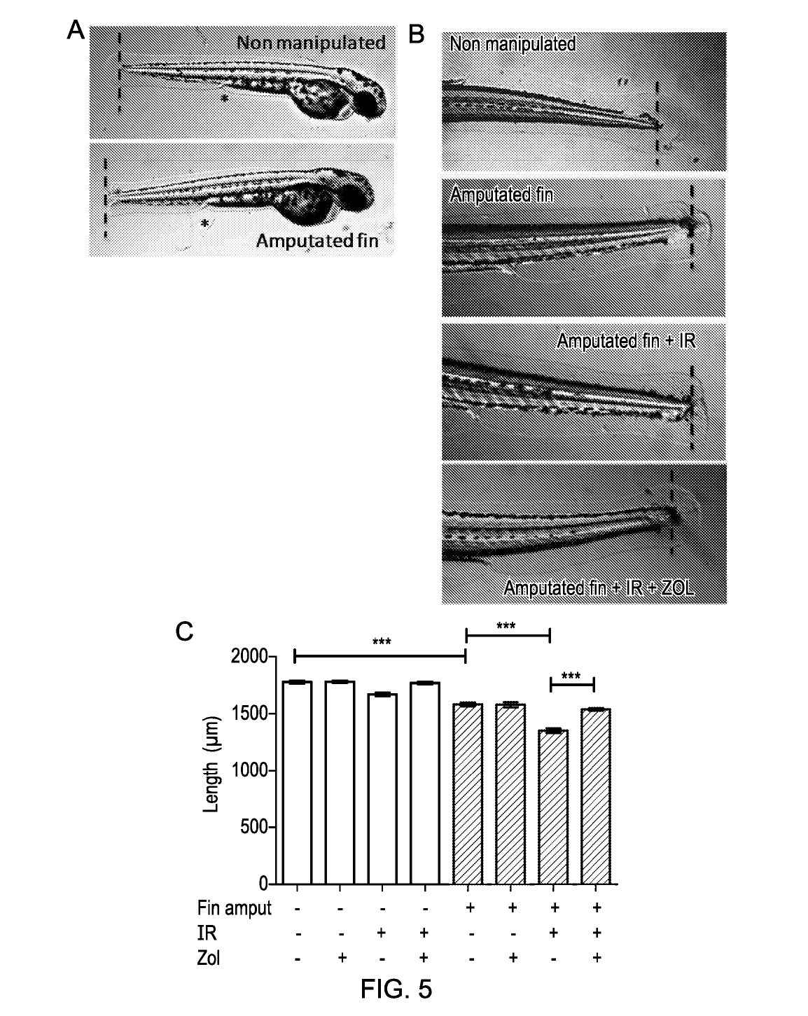

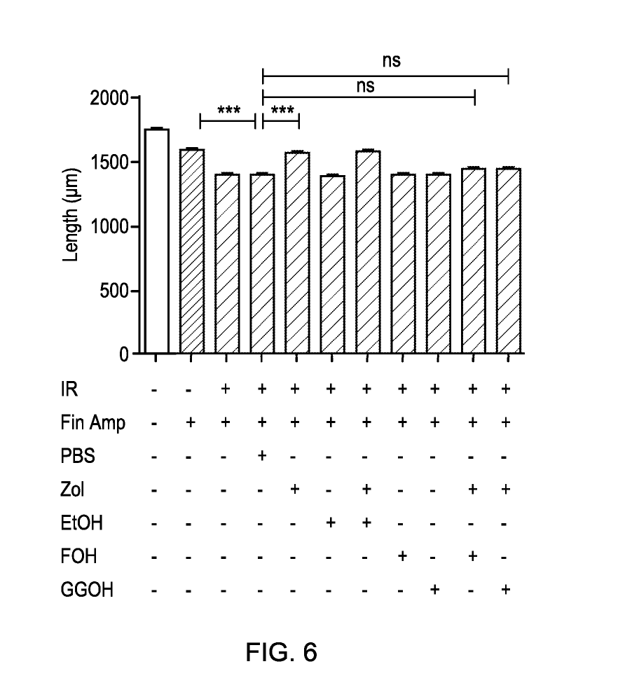

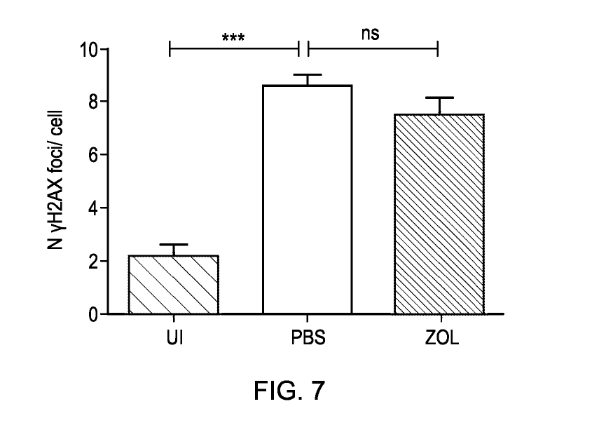

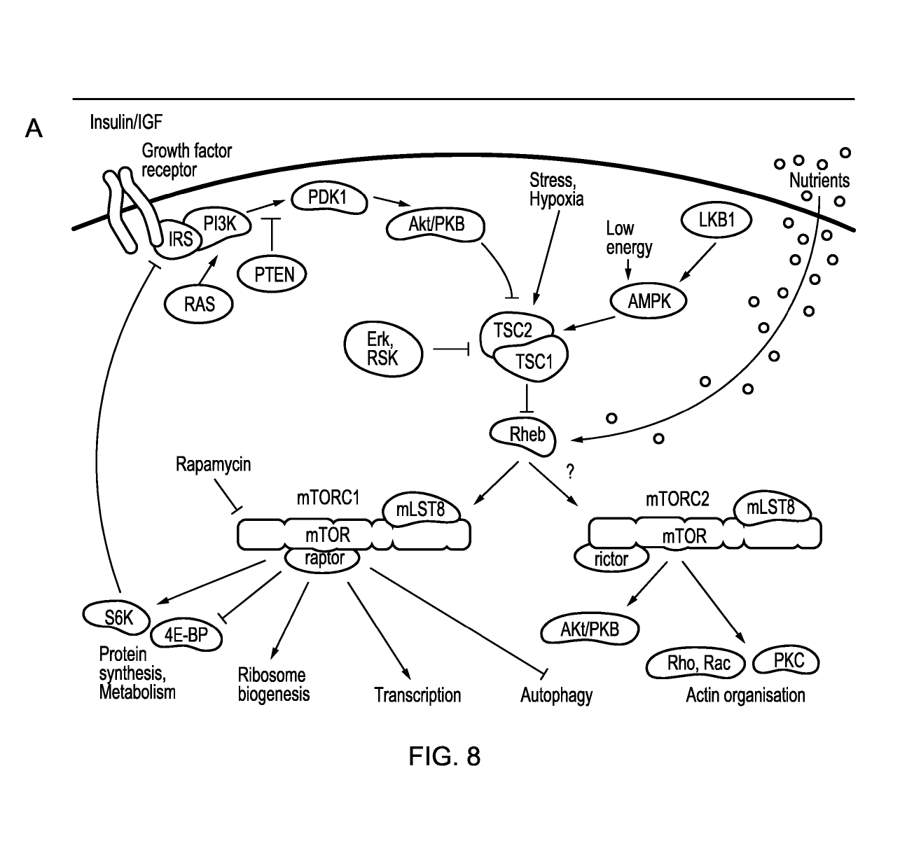

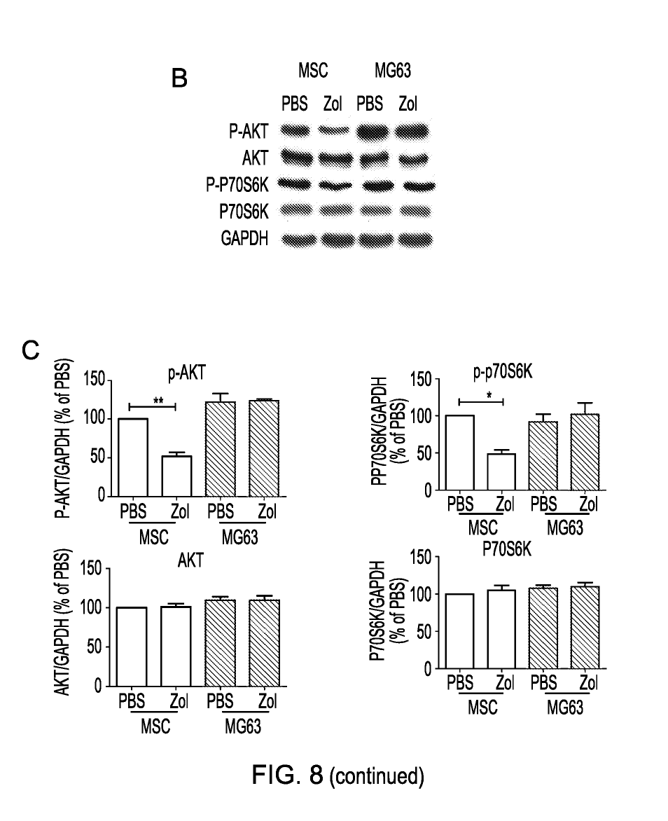

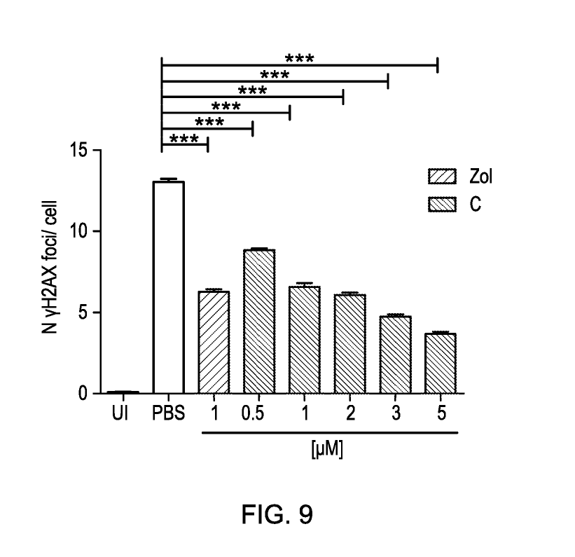

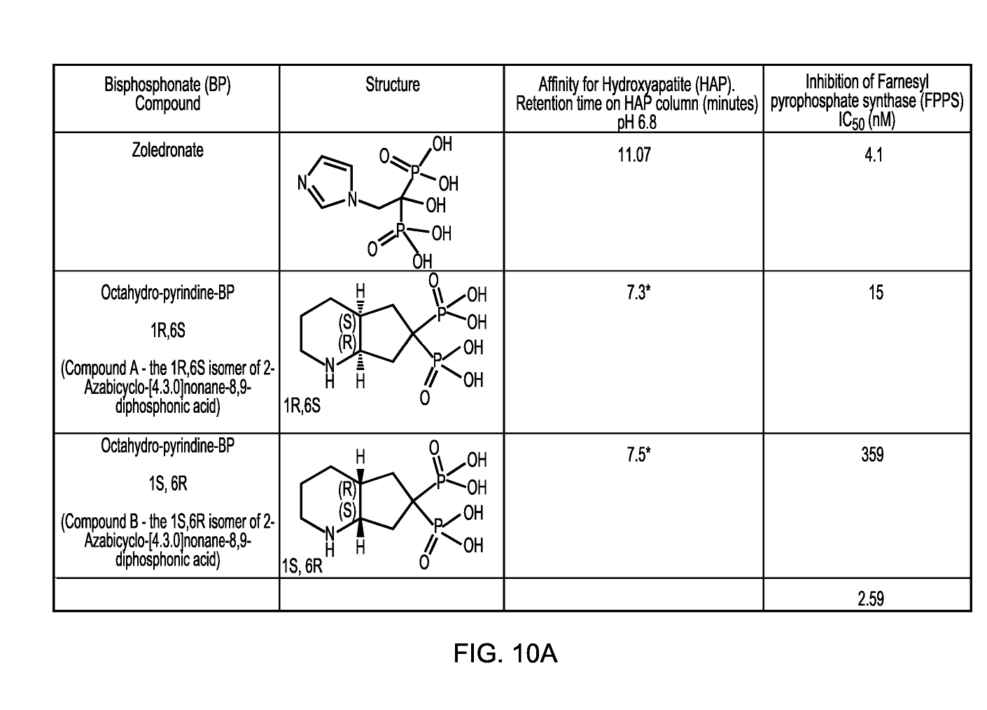

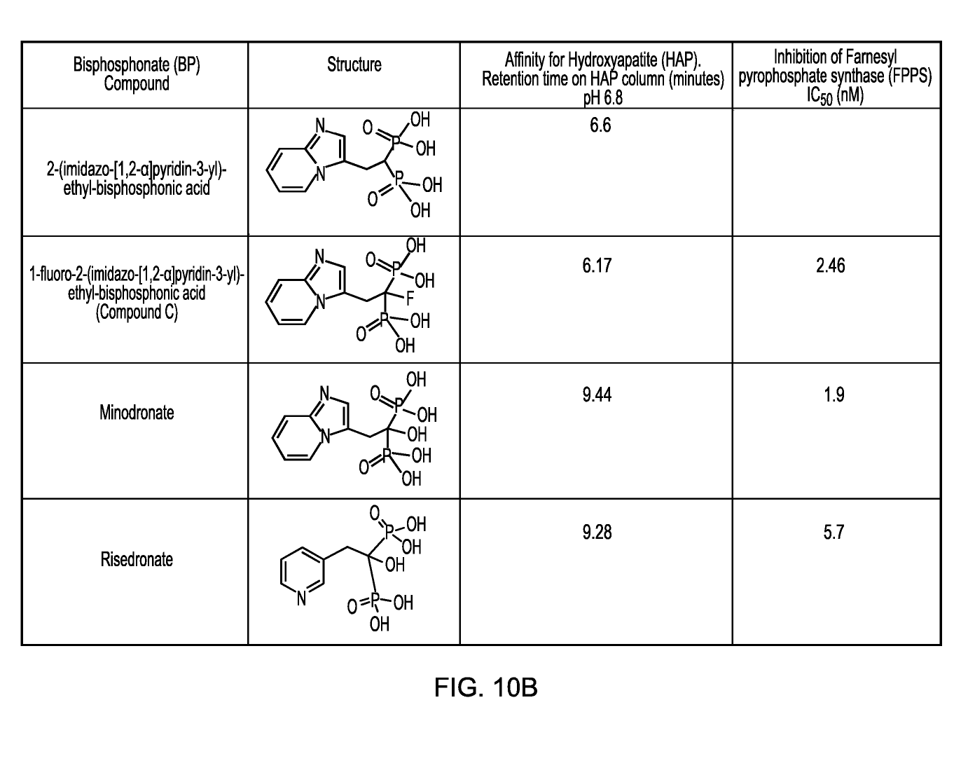

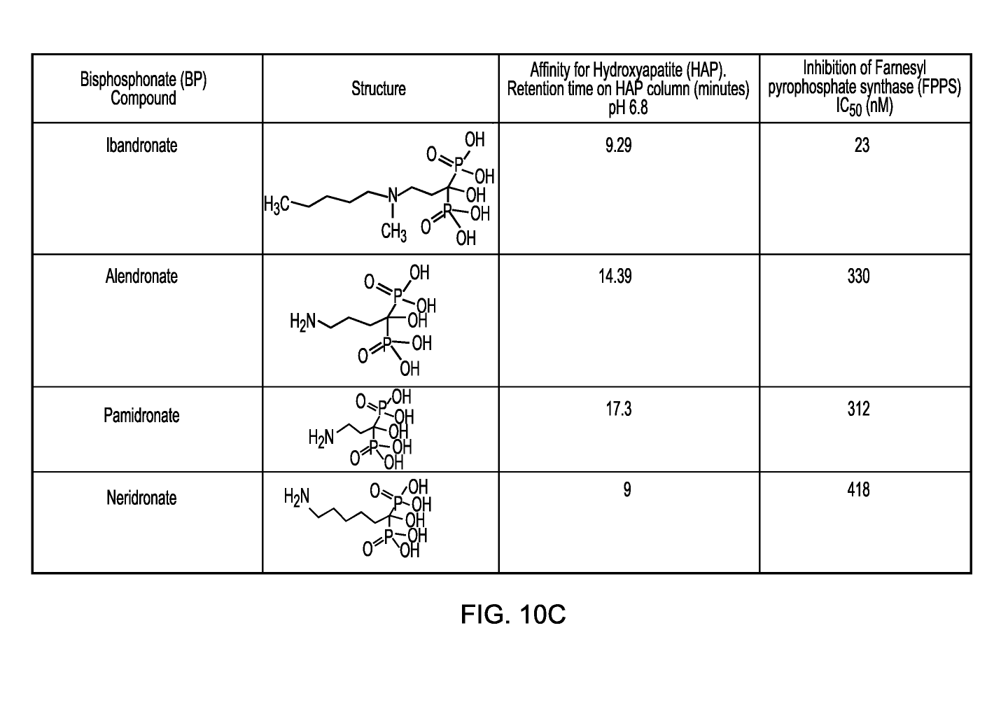

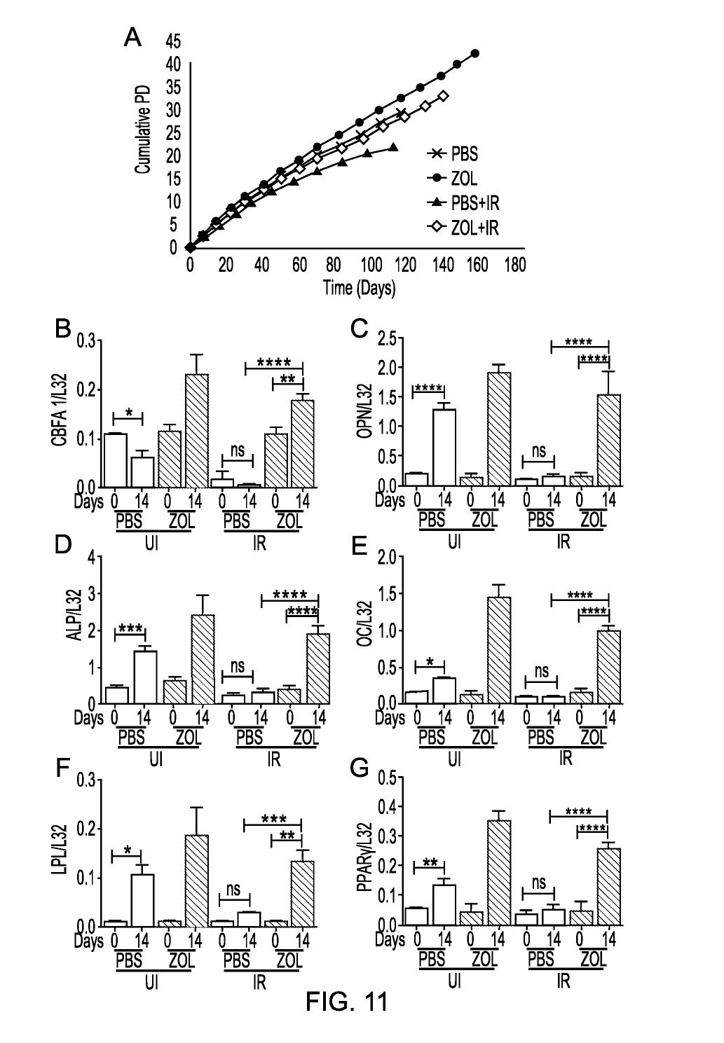

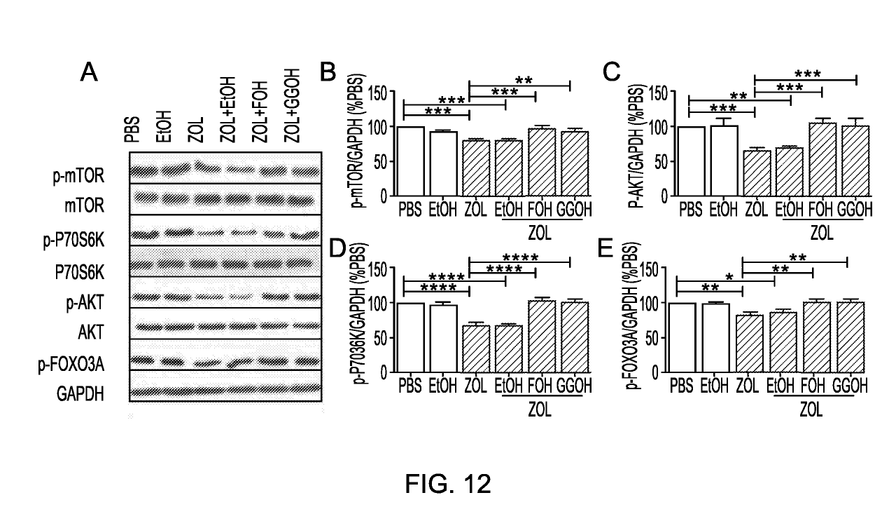

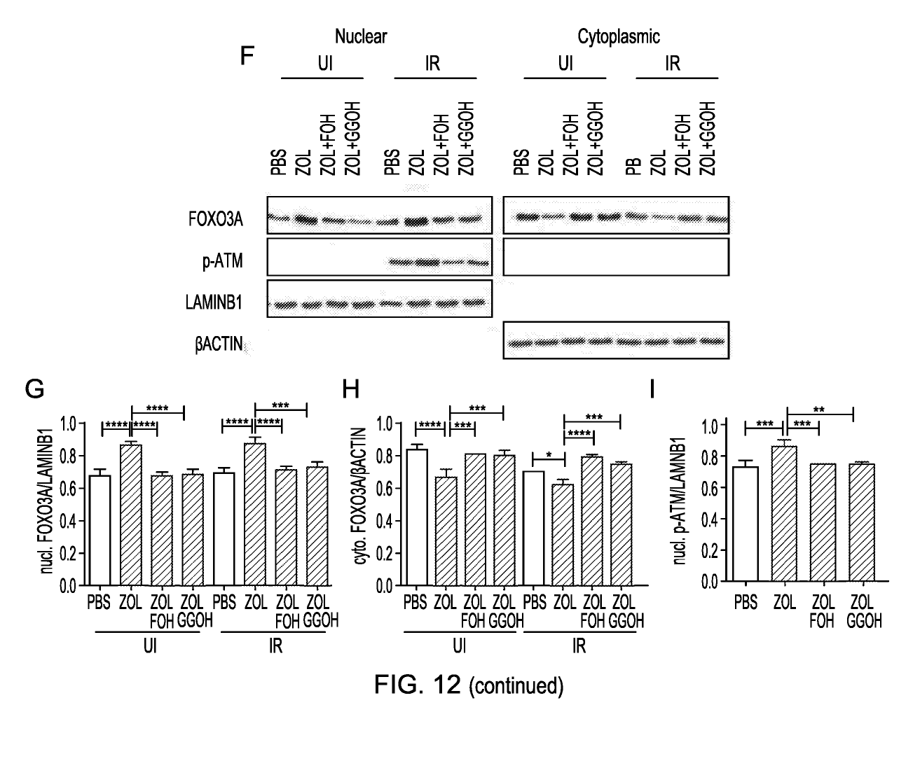

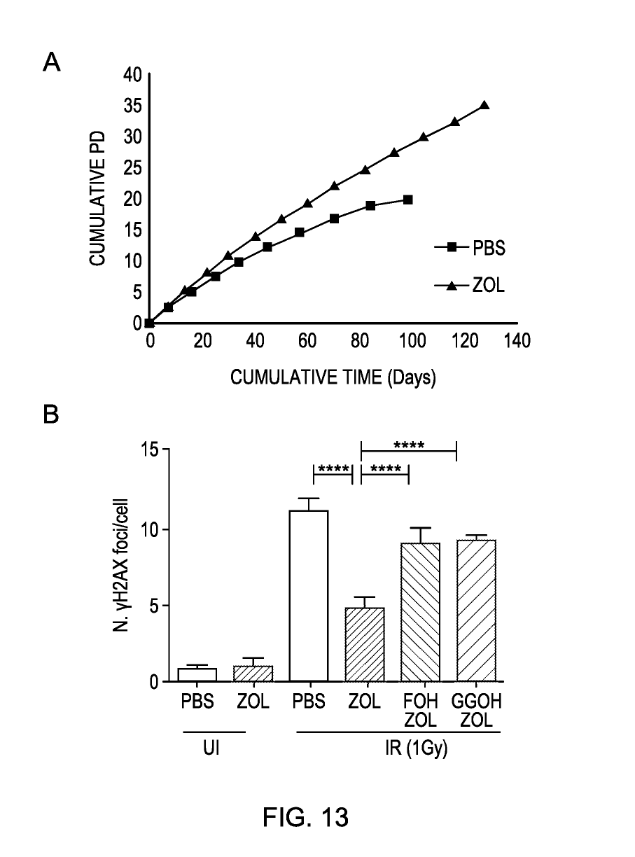

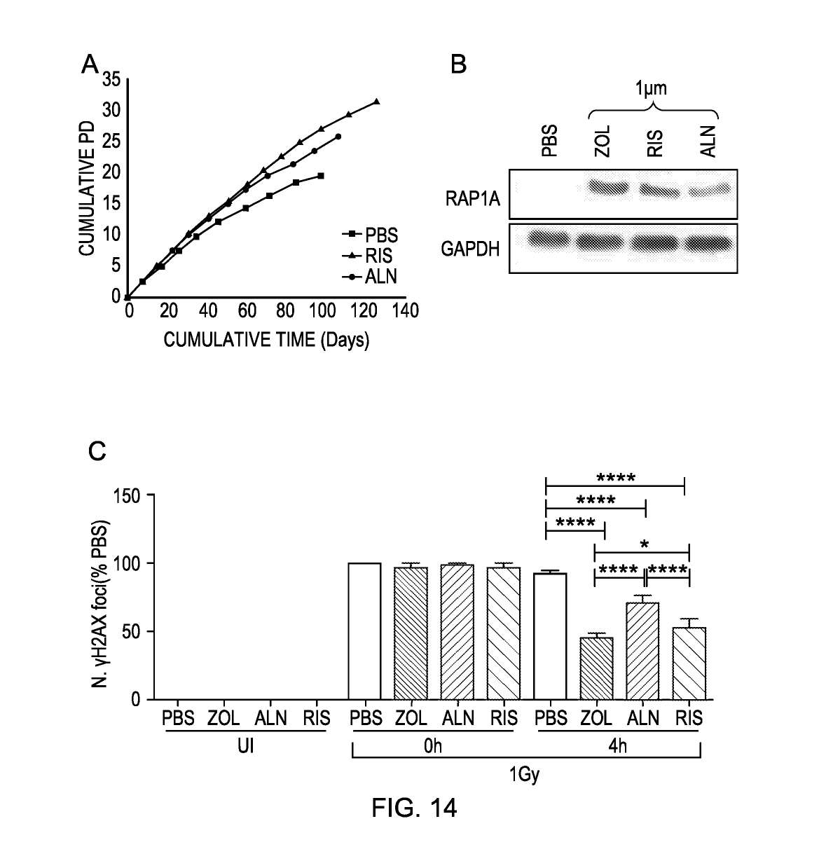

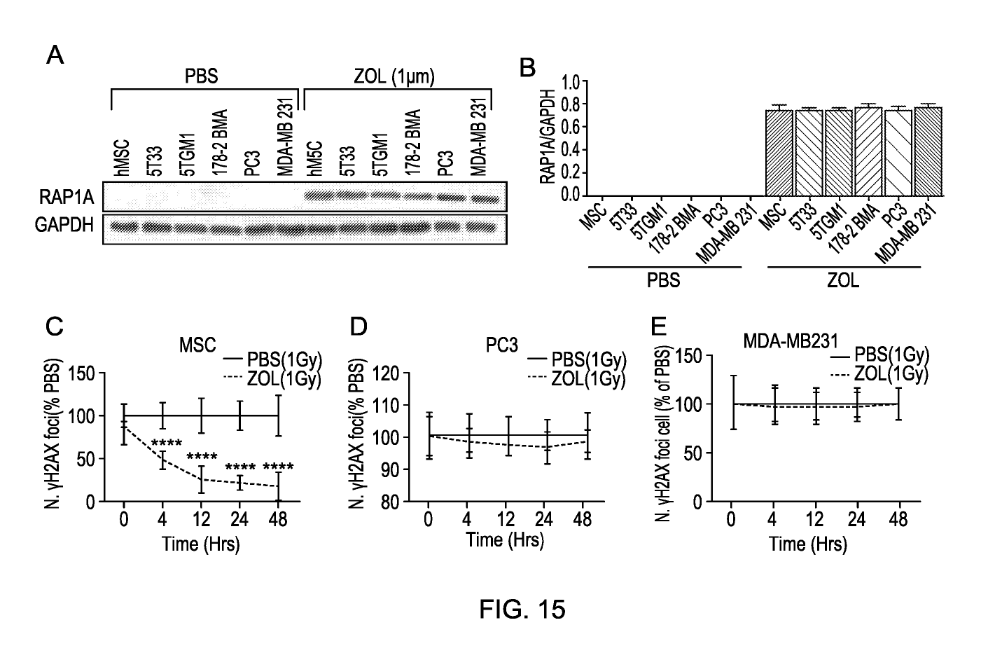

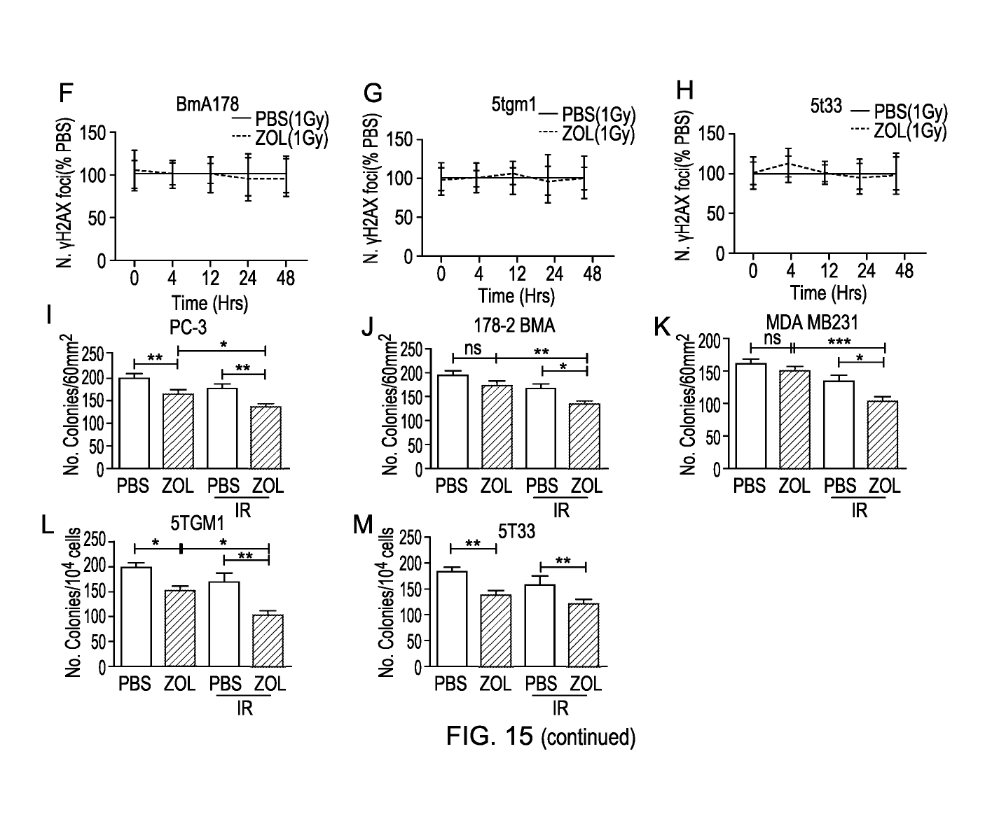

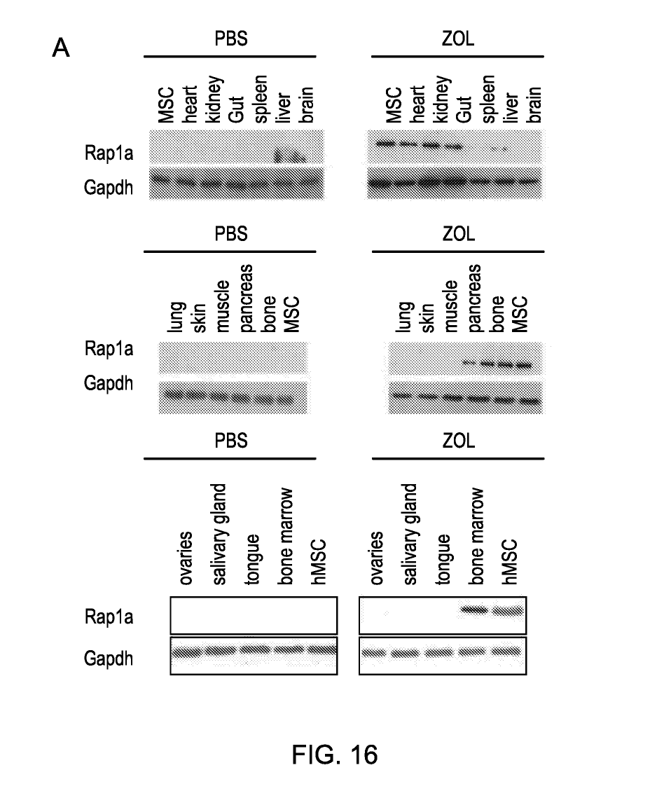

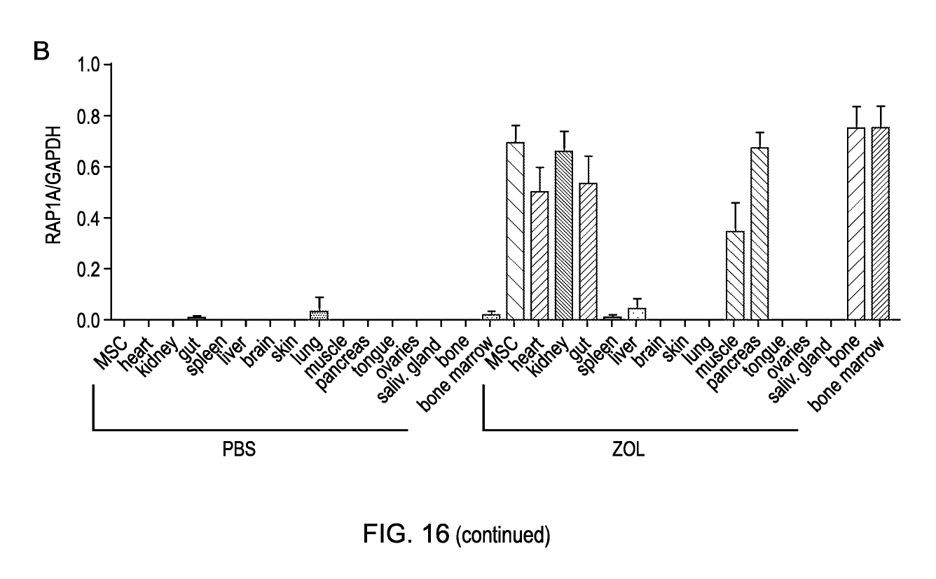

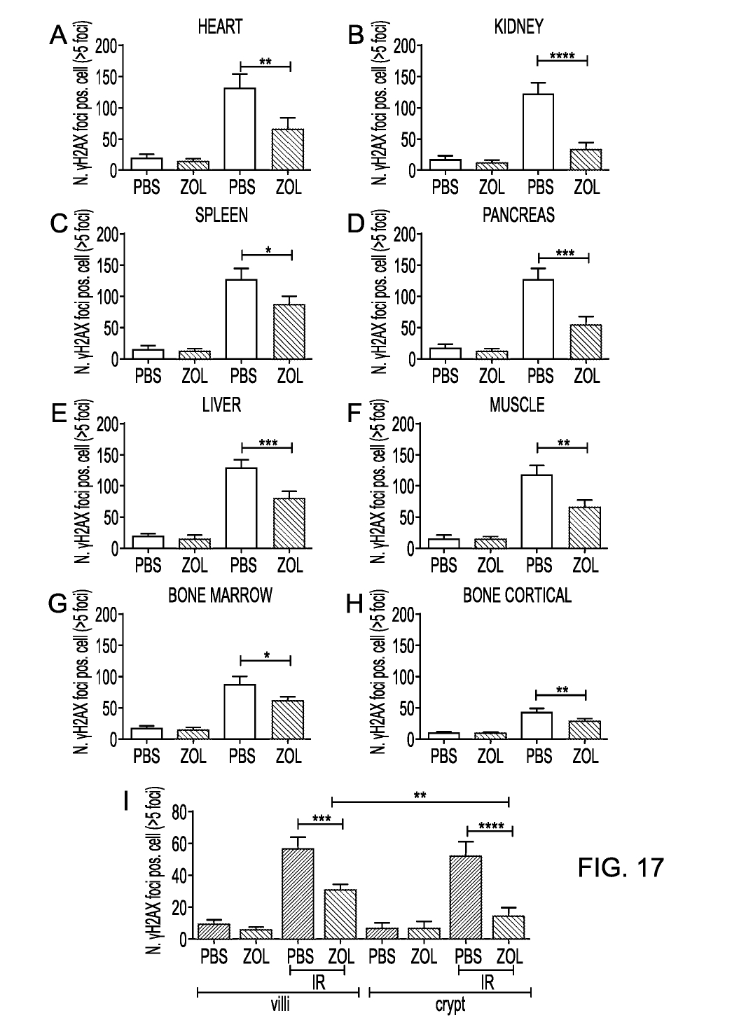

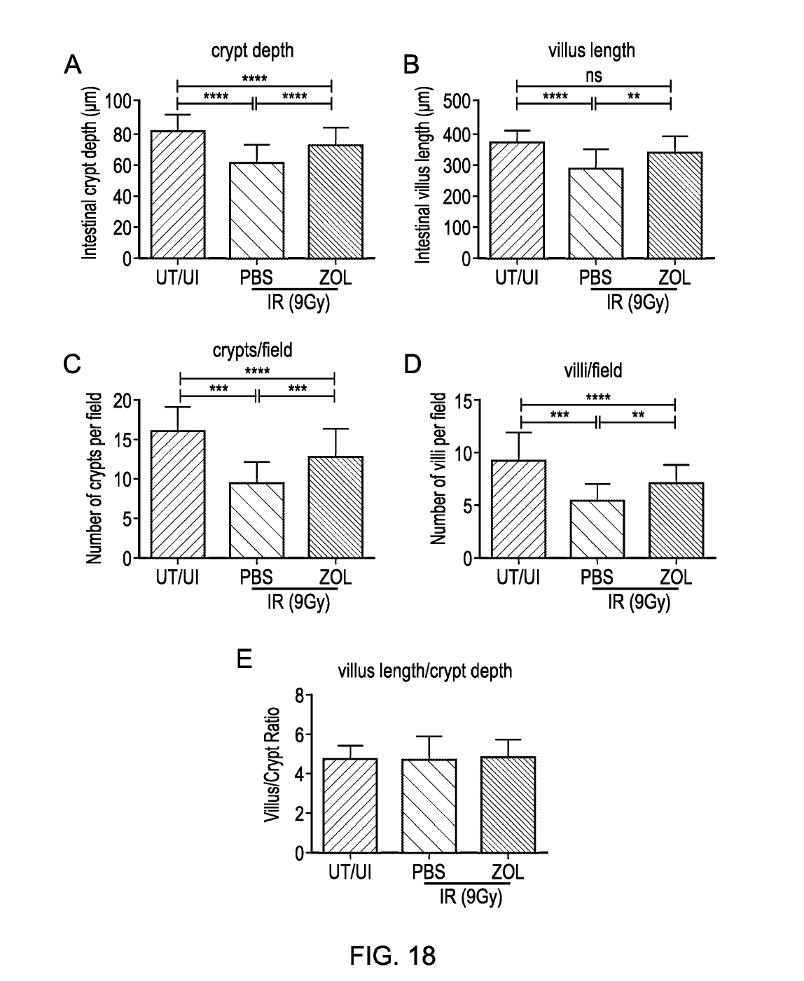

(A) A representative example of hMSC culture grown in presence or absence of Zol shows cumulative population doubling of hMSC with time in culture. Cultures (n=3) grown in PBS senesced after 21-27 population doublings (PDs) (square symbols) whereas cultures grown in presence of Zol were still proliferating after 29-36 PDs (triangle symbols) (This is a repeat of FIG. 1A).

(B) Clonogenic ability of hMSC at passage 8 showed a significant increase in the number of clonogenic progenitors (Colony Forming Unit-Fibroblast) in hMSC cultured in the presence of Zol in comparison to PBS control when seeded at low density in hMSC medium and left to grow for 14 days at 37 C in 5% CO2 in air. (This is a repeat of FIG. 1B)

(C-H) Human MSC exposed to osteogenic (C-F) and adipogenic (G-H) differentiation supplements for 14 days and assessed for expression of osteogenic differentiation markers (C) CBFA-1, (D) osteopontin(OPN), (E) alkaline phosphatase(ALP) (F) osteocalcin (OC), and adipogenic differentiation markers (G) Lipoprotein lipase (LPL) and (H) peroxisome proliferator-activated receptor γ (PPAR-γ). All markers were normalised to ribosomal protein L-32.

(I) Incidence of DNA damage foci enumerated at passage 3 (early) and p10 (late) in hMSC in the presence or absence of Zol show accumulation of DNA damage to be significantly higher in untreated hMSC.

(J) A representative example of γH2AX foci (green) in DAPI stained nuclei (blue) in PBS and ZOL treated hMSC at early (Ji-ii) and late (Jiii-iv) passage.

All data are presented as mean±SD and analysed by t-tests or for multiple comparisons by one way ANOVA with Bonferroni multiple comparison post-hoc test *p<0.05, **p<0.01, ***p<0.001, ****p<0.0001.

FIG. 2: Zoledronate (Zol) enhanced DNA repair in hMSC exposed to irradiation and rescues their clonogenic ability.

(A) A representative example of hMSC exposed to 1Gy irradiation and stained for phosphorylated γH2AX 4h after. Panels i, iii and v represent cells with γH2AX DNA damage foci in green. Panels ii, iv, vi are the same cells with overlaid DAPI nuclear staining (blue). UI (panels i and ii) is hMSC not irradiated and cultured in hMSC medium, PBS (panels iii and iv) are cells irradiated in absence of Zol, Zol (panels v and vi) are cells irradiated in the presence of Zol at 1 μM

(B) Number of γH2AX DNA damage foci in hMSC cultured in MSC medium and not irradiated (UI), hMSC irradiated in absence of Zol (PBS) and hMSC irradiated in presence of Zol at 1 μM (n=3) immediately after irradiation (0 h) and 4, 12, 24 hours after irradiation. A significant decrease in the presence of DNA damage foci was observed when hMSC were irradiated in presence of Zol. A significant decrease was also observed when hMSC were exposed to 3 and 5Gy of irradiation following the same protocol (data not shown).

(C) hMSC seeded at low density and exposed to irradiation in presence or absence of Zol were left to grow for 14 days at 37° C., 5% CO₂ in air. The number of CFU-F was evaluated to determine the clonogenic ability of the cells. As expected hMSC exposed to irradiation (1, 3 and 5Gy) showed a significant decrease in the number of CFU-F. However no significant difference was found between hMSC irradiated and non irradiated in presence of Zol, even at 5Gy, suggesting rescue of their clonogenic ability. Data are expressed as mean±SEM and were analysed by one way ANOVA and Bonferroni post-test for multiple comparison *p<0.05, **p<0.01, ***p<0.001

FIG. 3: Zoledronate (Zol) enhances DNA repair by inhibiting the mevalonate pathway in MSC.

(A) A schematic representation of the mevalonate pathway and blocking of FPP synthase by Bisphosphonates (BPs) including Zol. Highlighted with circles are where farnesol (FOH) and Geranylgeraniol (GGOH) act to reverse the inhibition.

(B) Expression of unprenylated Rap1A in hMSC exposed to increasing doses of Zol;

(C) Number of γH2AX DNA damage foci in response to different doses of Zol.

(D) Number of γH2AX DNA damage foci in hMSC not irradiated (UI) or exposed to 1Gy in the presence or absence of Zol and with the addition of Farnesol (FOH) or geranylgeraniol (GGOH).

Ethanol (EtOH) was added with Zol in the same amount used to dissolve GGOH and FOH as control. Data are expressed as mean±SEM and were analysed by one way ANOVA and Bonferroni post-test for multiple comparison *p<0.05, **p<0.01, ***p<0.001

FIG. 4: Only bisphosphonates with high inhibitory affinity for FPP synthase enhance DNA repair in mesenchymal stem cells following irradiation.

New bisphosphonates (BPs) isomers Compound A (high affinity for FPP synthase) and Compound B (low affinity for FPP synthase) were also used. The number of γH2AX DNA damage foci was measured in hMSC cultured in MSC medium and not irradiated (UI), hMSC irradiated in absence of BPs (PBS) and hMSC irradiated in presence of Zol, Compound A, or B at 1 μM (n=3) 4, hours after irradiation. A significant decrease in the presence of DNA damage foci was observed when hMSC were irradiated in presence of Zol or Compound A but not B. Data are expressed as mean±SEM and were analysed by one way ANOVA and Bonferroni post-test for multiple comparison *p<0.05, **p<0.01, ***p<0.001

FIG. 5: Zoledronate enhances tail regeneration in zebrafish embryos exposed to 5 Gy irradiation.

(A) Top panel is a representative example of zebrafish embryo at 72 h postfertilization (hpf); bottom panel is a representative example of zebrafish 72 hpf following fin amputation.

(B) First panel from the top is a representative example of Zebrafish 120 hpf. Second panel is a representative example of zebrafish 120 hpf which has undergone fin amputation at 48 hpf, Third panel is a representative example of zebrafish at 120 hpf which has been irradiated (IR, 5Gy) and has undergone fin amputation 48 hpf. The fourth panel is a representative example of zebrafish at 120 hpf which has been irradiated and has undergone fin amputation in the presence of Zol (1 μM) at 48 hpf.

(C) Regeneration of the caudal fin at 120 hpf. Fins were amputated at 72 hpf in presence or absence of irradiation at 5Gy (IR) and in presence or absence of zoledronic acid at 1 μM (Zol) and the length of the fin measured (n=15/group). Dashed lines indicate the plane of amputation. * indicates the starting reference point for the measurement of the fin length. Similar data have been obtained at 1 and 3 Gy (data not shown). Data represent mean±SEM and were analysed by one way ANOVA and Bonferroni post-test for multiple comparison *p<0.05, **p<0.01, ***p<0.001. The same experiment was repeated at 1 and 3 Gy with the same outcome (data not shown).

FIG. 6: Zoledronate (Zol) enhances tail regeneration by inhibiting the mevalonate pathway in zebrafish embryos.

Regeneration of the caudal fin at 120 hpf. Fins were amputated at 48 hpf in presence or absence of irradiation at 1Gy (IR), in presence or absence of zoledronic acid at 1 μM (Zol) and with the addition of farnesol (FOH) or geranylgeraniol (GGOH). Ethanol (EtOH) was added with Zol in the same amount used to dissolve GGOH and FOH as control. The length of the fin was measured at 120 hpf (n=15/group). Data represent mean±SEM and were analysed by one way ANOVA and Bonferroni post-test for multiple comparison *p<0.05, **p<0.01, ***p<0.001.

FIG. 7: Zoledronate (Zol) does not enhance the DNA repair capacity in 5T33 Multiple Myeloma line.

5T33MM were exposed to 1 μM Zol for 3 days prior to irradiation at 1Gy and assessment of γH2AX 4 h later (Zol). UI, are hMSC non irradiated, PBS, are hMSC irradiated in absence of Zol. Data are expressed as mean±SEM and were analysed by one way ANOVA and Bonferroni post-test for multiple comparison *p<0.05, **p<0.01, ***p<0.001

FIG. 8: Zoledronate acts by inhibiting the mTOR pathway in mesenchymal stem cells but not in cancer cells.

(A) A schematic representation of the mTOR pathway;

(B) A representative example of human mesenchymal stem cells (MSC) and human osteosarcoma cell line MG63 cultured in presence or absence of Zol (1 μM) for 72 h and assessed for the expression of phosphorylated (Ser 473) AKT, AKT, phosphorylated p70S6K p70S6K and GAPDH by western blot;

(C) Quantitation of the expression of the same proteins normalised to GAPDH in hMSC assessed by western blot and analysed using ImageJ software (n=3). Data represent mean±SEM and were analysed by one way ANOVA and Bonferroni post-test for multiple comparison *p<0.05, **p<0.01, ***p<0.001.

FIG. 9: A novel BP (Compound C) with lower affinity for bone mineral enhanced DNA repair in hMSC in a similar way to Zoledronate.

Enumeration of the number of γH2AX DNA damage foci in hMSC cultured in MSC medium and not irradiated (UI), hMSC irradiated in absence of Zol (PBS), hMSC irradiated in presence of Zol at 1 μM and hMSC in presence of Compound C at increasing concentrations (n=3) 4 hours after irradiation. A significant increase in the number of DNA damage foci was observed when hMSC were irradiated in absence of Zol. In presence of Zol a significant decrease in the number of DNA damage foci was observed. Irradiation in presence of Compound C enhances DNA repair in a dose dependent manner. Data represent mean±SEM and were analysed by one way ANOVA and Bonferroni post-test for multiple comparison *p<0.05, **p<0.01, ***p<0.001.

FIG. 10: Chemical structures of some bisphosphonate compounds.

The Table lists a number of bisphosphonate compounds, together with their structures. Also provided is an indication of their affinity for hydroxyapatite, and inhibition of farnesyl pyrophosphate synthase (FPPS).

FIG. 11 Zoledronate (Zol) rescues hMSCs ability to proliferate and differentiate following exposure to irradiation.

(A) Representative example of a growth curve showing number of cumulative population doublings (PD) of hMSC with time in culture. Cultures were either left non-irradiated or irradiated at 3 Gy and grown in the presence (non-irradiation, circle; plus irradiation, diamond) or absence of Zol (non-irradiation, cross; plus irradiation, triangle) for 3 days, and 12h later cultures were washed free from Zol and expanded in hMSC medium (n=3).

(B-G) Human MSC treated as described in (A) and at passage 9 exposed to osteogenic and adipogenic differentiation supplements respectively. Cultures exposed to osteogenic supplements were assessed for the expression of osteogenic differentiation markers (B) core binding factor subunit alphal (CBFA1), (C) osteopontin (OPN), (D) alkaline phosphatase (ALP), (E) osteocalcin (OC). Cultures exposed to adipogenic supplements were assessed for adipogenic differentiation markers

(F) Lipoprotein lipase (LPL) and (G) peroxisome proliferator-activated receptor γ (PPAR γ). All markers were normalised to ribosomal protein L-32 (n=3).

Data expressed as mean ±SD and analysed by one way ANOVA and Bonferroni post-hoc test for multiple comparisons *p<0.05, **p<0.01, ***p<0.001, ****p<0.0001.

FIG. 12 Zoledronate (Zol) mediates an enhanced repair response to DNA damage via inhibition of mTOR signaling.

(A-E) A representative example of expression of p-mTOR (Ser2448), mTOR, p-P70S6K (Thr421/Ser424), P70S6K, p-AKT (Ser473), AKT and p-FOXO3A (Ser318/321) by western blot analysis in hMSC exposed to zoledronate (ZOL) alone or in combination with farnesol (FOH) or geranylgeraniol (GGOH). PBS was added in the same amount than Zol, and ethanol (EtOH) was added in the same amount than GGOH and FOH as controls. Protein expression was normalised to the expression of glyceraldehyde 3-phosphate dehydrogenase (GAPDH). Quantitation of phosphorylated proteins (B) p-mTOR, (C) p-AKT, (D) p-P70S6K and (E) p-FOXO3a using imageJ (n=3)

(F-I) A representative example of expression of nuclear and cytosolic FOXO3A and p-ATM (Ser1981) in non-irradiated hMSC (UI) and in hMSC 10 minutes after irradiation (IR) in the presence or absence of Zol normalised to expression levels of LaminB1 and βACTIN respectively. (C) Quantification of (G) nuclear and (H) cytosolic FOXO3A and (I) nuclear p-ATM in non-irradiated hMSC and in hMSC 10 minutes after irradiation in the presence or absence of Zol using imageJ (n=3)

Data are expressed as mean±SD and were analysed by one way ANOVA and Bonferroni post-hoc test for multiple comparisons *p<0.05, **p<0.01, ***p<0.001, ****p<0.0001.

FIG. 13 Zoledronate (Zol) extends lifespan of normal human dermal fibroblasts and enhances DNA repair ability following irradiation.

(A) A representative example of human dermal fibroblasts culture grown in presence or absence of Zol shows cumulative population doubling of human dermal fibroblasts with time in culture. Cultures (n=3) grown in PBS senesced after 17-21 population doublings (PDs) (square symbols) whereas cultures grown in presence of Zol were still proliferating after 27-34 PDs (triangle symbols).

(B) Number of yH2AX DNA damage foci in human dermal fibroblasts (n=4 cultures) not irradiated (UI) or exposed to irradiation (IR) 1Gy in the presence or absence of Zol (1 μM) and with the addition of Farnesol (FOH) or geranylgeraniol (GGOH).

Data are expressed as mean±SD and were analysed by one way ANOVA and Bonferroni post-hoc test for multiple comparison *p<0.05, **p<0.01, ***p<0.001, ****<0.0001.

FIG. 14 Bisphosphonate Alendronate (ALN) and Risedronate (RIS) extend hMSC lifespan and enhance repair of DNA damage following irradiation as indicated by a reduction in the number of yH2AX DNA damage foci.

(A) A representative example of hMSC culture grown in presence or absence of Alendronate (ALN) or Risedronate (RIS) shows cumulative population doubling of hMSC with time in culture. Cultures (n=3) grown in PBS senesced after 21-25 population doublings (PDs) (square symbols) whereas cultures grown in presence of RIS stopped proliferating after 29-34 PDs (triangle symbols) from the start of the treatment and cultures grown in presence of ALN (circle symbols) stopped proliferating after 27-30 PDs.

(B) Expression of unprenylated Rap1A in hMSC exposed to ALN or RIS at 1 ρM (n=3).

(C) The number of yH2AX DNA damage foci was measured in hMSC cultured in MSC medium and not irradiated (UI), hMSC irradiated in absence of BPs (PBS) and hMSC irradiated in presence of ZOL, ALN, or RIS at 1 μM (n=3) 0 and 4 hours after irradiation.

Data are expressed as mean±SD and were analysed by one way ANOVA and Bonferroni post-hoc test for multiple comparison *p<0.05, **p<0.01, ***p<0.001, ****<0.0001.

FIG. 15 Zoledronate (Zol) does not enhance the DNA repair capacity in murine and human cancer lines despite inhibition of mevalonate pathway.

(A) A representative example of western blot analysis of human mesenchymal stem cells (MSC) and human and murine prostate cancer cell lines (human: PC3, murine 178-2 BMA), human breast cancer cell line (MDA-MB231), murine multiple myeloma cancer lines (5TGM1 and 5T33) cultured in presence or absence of Zol (1 μM) for 72 h and assessed for the expression of unprenylated RAP1A and GAPDH.

(B) Quantitation of the expression of the RAP1A normalised to GAPDH in hMSC assessed by western blot and analysed using ImageJ software (n=3).

(C-H) Number of γH2AX foci enumerated in (C) hMSC, (D) PC3, (E) 178-2 BMA, (F) MDA-MB231,

(G) 5TGM1 and (H) 5T33 lines following irradiation (1Gy) in the presence or absence of Zol (1pM) and assessed at 0, 4, 12, 24 and 48 h post irradiation (n=3).

(I-M) Clonogenic assays obtained from (I) PC3, (J) 178-2 BMA, (K) MDA-MB231, (L) 5TGM1 and (M) 5T33 lines cultures seeded at low density and exposed to irradiation (1 Gy) in the presence or absence of Zol and left to grow for 14 days at 37 C, 5% CO2 in air (n=3);

Data represent mean±SD and were analysed by one way ANOVA and Bonferroni post-hoc test for multiple comparison *p<0.05, **p<0.01, ***p<0.001, ****<0.0001.

FIG. 16 Zoledronate (Zol) treatment results in increased levels of Rap1A unprenylation

(A) A representative example of western blot analysis of tissues (heart , kidney, intestines, spleen, liver, brain, skin, lung, muscle, pancreas, bone, ovaries, salivary gland, tongue, bone marrow) obtained from C57B16/J mice treated with either PBS or Zol (125 μg/kg, i.p.) for 3 days and assessed for expression of unprenylated RAP1A and GAPDH.

(B) Quantitation of the level of expression of unprenylated RAP1A normalised to GAPDH in murine tissues assessed by western blot and analysed using ImageJ software (n=6mice/group).

Data represent mean±SD and were analysed by one way ANOVA and Bonferroni post-hoc test for multiple comparison *p<0.05, **p<0.01, ***p<0.001, ****<0.0001.

FIG. 17 Zoledronate (Zol) treatment results in decreased levels of DNA damage following irradiation in murine tissues as indicated by the numbers of yH2AX foci observed.

(A-L) Number of γH2AX foci enumerated in tissues from C57B16/J either un-irradiated (UI) or following irradiation (3Gy) in the presence or absence of Zol (125 μg/kg, i.p. for 3 days) and assessed at 12 h post irradiation (n=6 mice/group). (A) heart, (B) kidney, (C) spleen, (D) pancreas, (E) liver (F) muscle (G) bone marrow (H) bone and (L) intestine villi and crypts.

Data represent mean±SD and were analysed by one way ANOVA and Bonferroni post-hoc test for multiple comparison *p<0.05, **p<0.01, ***p<0.001, ****<0.0001.

FIG. 18 Zoledronate protects intestinal crypt and villi following irradiation in C57BI6/J mice

C57BL6/J mice (n=3/group) injected with ZOL (125 μg/kg, i.p.) or PBS 3 days prior to 9Gy irradiation (IR), were sacrificed for intestine regeneration assessment 4 days later.

(A) Intestinal crypt depth and (B) villi length were measured ad found to be improved by treatment with Zol

In each animal the number of intestinal (C) crypts and (D) villi were enumerated in an area of 0.525 mm² at three different levels and the average of all sections in each animal was taken (n=3 mice/group).

(E) It represents villi atrophy by expression of the villus length to crypt depth ratio

Data represent mean±SD and were analysed by one way ANOVA and Bonferroni post-hoc test for multiple comparison *p<0.05, **p<0.01, ***p<0.001, ****<0.0001.

### DETAILED DESCRIPTION OF THE INVENTION

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Throughout the description and claims of this specification, the words “comprise” and “contain” and variations of the words, for example “comprising” and “comprises”, mean “including but not limited to”, and are not intended to (and do not) exclude other moieties, additives, components, integers or steps. However, the words are intended to encompass “consisting of” and “consisting essentially of”.

Unless otherwise noted, technical terms are used according to conventional usage. Definitions of common terms in molecular biology may be found in Benjamin Lewin, Genes V, published by Oxford University Press, 1994 (ISBN 0-19- 854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All publications, patents and patent applications mentioned in this specification are herein incorporated by reference into the specification to the same extent as if each individual publication, patent or patent application was specifically and individually indicated to be incorporated herein by reference.

This disclosure references various Internet sites. The contents of the referenced Internet sites are incorporated herein by reference as of 27 Mar. 2013.

All references to “detectable” or “detected” are as within the limits of detection of the given assay or detection method.

As described in the present Examples, the present inventors have identified surprising new cytoprotective properties of bisphosphonate (BP) compounds. The invention relates to various uses of BP compounds as cytoprotectants, and to associated methods and products.

As used herein, a cytoprotectant refers to an agent (or combination of agents) which promotes cell survival. Thus, cells treated with or exposed to the cytoprotectant under suitable conditions, demonstrate increased survival compared to cells not treated or exposed in the same way.

The invention is concerned in particular with the ability of BP compounds to promote cell survival by protecting cells from damage, in particular, DNA damage. Protection of cells against DNA damage as used herein generally includes protection against accumulation of DNA damage in the cells.

Damage may, for example, be induced in cells by exposure to cell-damaging radiation and/or to one or more cell-damaging chemical agents, as described herein. The protective effects of the BP compounds are seen particularly in non-cancerous cells. The invention finds particular use in protecting non-cancerous cells from the effects of exposure to damaging radiation and/or chemical agents during radiotherapy or chemotherapy treatment for cancer.

BP Compounds

Any suitable bisphosphonate compound (BP compound) may be used in the invention. Many BP compounds are known in the art, and are sometimes also referred to as diphosphonate compounds. For example, a number of BP compounds are reviewed in Ebetino, F H et al (2011) Bone 49, 20-33.

Reference to a BP compound or use of a BP compound herein may in general (and unless the context requires otherwise) also refer to a pharmaceutically acceptable salt or solvate of the BP compound or use of such a salt or solvate.

Reference to a BP compound or use of a BP compound herein may in general (and unless the context requires otherwise) also refer to a pro-drug of a bisphosphonate compound, or use of such a pro-drug.

A BP compound as referred to herein may comprise a compound which is an analog of endogenous pyrophosphate whereby the central oxygen is replaced by carbon.

A BP compound may have a general formula (OH)₂P(O)CR¹R²P(O)(OH)₂ (Formula I). Such BP compounds share a common backbone P—C—P, in which two phosphonate groups (PO₃) are covalently linked to C. R¹ and R² typically represent a short side chain (e.g. H or OH) and a long side chain respectively. The term bisphosphonate may in one aspect include prodrugs thereof and amino bisphosphonates.

A BP compound for use in the invention may comprise a nitrogen-containing side chain (e.g. R2 side chain in Formula I) and may be referred to as a nitrogen-containing bisphosphonate compound (N—BP compound).

A BP compound for use herein may comprise any suitable BP compound (such as a N—BP compound), which is licensed for human use.

For example, BP compounds in clinical use may include: Alendronate, Clodronate, Etidronate, Ibandronate, Risedronate, Tiludronate, Pamidronate, Zoledronate, Neridronate, and Minodronate, or a pharmaceutically acceptable salt or solvate thereof, such as any of those referred to herein. Of these, Alendronate, Ibandronate, Risedronate, Pamidronate, Zoledronate, Neridronate, and Minodronate are N-BPs. Structures for a number of these compounds are set out in FIG. 10. BP compounds (or pharmaceutically acceptable salts or solvates thereof) prescribed (e.g. for oral or intravenous use) in the UK (www.mhra.gov.uk) include, for example, any of those in Table 1 below:

| BP compound | Examples of brand names |
|---|---|
| Alendronate | Fosamax ®, Fosavance ® |
| Clodronate (e.g. sodium | Bonefos ®, Loron ® |
| clodronate) | |
| Etidronate (e.g. disodium | Didronel ®, Didronel ® PMO |
| etidronate) | |
| Ibandronate | Bondronat ®, Bonviva ® |
| Risedronate (e.g. | Actonel ®, Actonel ® Once a |
| risedronate sodium) | Week |
| Tiludronate (e.g. disodium | Skelid ® |
| tiludronate) | |
| Pamidronate (e.g. disodium | Aredia ® |
| pamidronate) | |
| Zoledronic Acid | Aclasta ®, Zometa ® |

A number of BP compounds, including N—BPs, are undergoing study or are in development.

For example a number of BP compounds and preparation thereof are described in the following documents: U.S. Pat. No. 7,781,418 B2 (which describes Compound A herein (the 1R,6S isomer of 2-Azabicyclo-[4.3.0]nonane-8,9-diphosphonic acid) and Compound B herein (the 1S,6R isomer of 2-Azabicyclo-[4.3.0]nonane-8,9-diphosphonic acid)); US 7,781,418 B2 (which describes Compound A herein (the 1 R,6S isomer of 2-Azabicyclo-[4.3.0]nonane-8,9-diphosphonic acid) and Compound B herein (the 1S,6R isomer of 2-Azabicyclo-[4.3.0]nonane-8,9-diphosphonic acid)); U.S. Pat. No. 7,268,124 B2 (which describes BP compounds of general Formula I as presented in the document and which act as GGPP synthase inhibitors); US 2011/0237550 A1 (which describes 5-azaindole BP compounds of general Formula I as presented in the document); and US 2011/0230443 A1 (which describes imidazo[1,2-a]pyridinyl BP compounds of general Formula I as presented in the document). The content of each of these documents is hereby incorporated by reference, in particular, the contents describing BP compounds and the preparation thereof. US 2010/0240612 A1 describes prenylated bisphosphonates which may also find use in the present invention. The content of this document are hereby incorporated by reference, in particular, the contents describing BP compounds and the preparation thereof.

Further examples of BP compounds, namely, phenylalkyl-imidazole-bisphosphonate compounds, are described in WO 2010/076258. The compounds have general Formula I as presented in the document. The contents of the document, in particular the contents describing the BP compounds and the preparation thereof, are hereby incorporated by reference.

A BP compound, for example, an N—BP compound, for use in the invention may,inhibit one or more steps in the mevalonate pathway (FIG. 3A) that generate isoprenoids.

A BP compound may, for example, have a high inhibitory potency on, the farnesyl pyrophosphate synthase enzyme (FPPS). Examples of BP compounds having high affinity include, for example, Compound A In one example, a BP compound for use herein may have an inhibitory potency on, FPPS, which is at least that of Compound A. Methods for determining inhibitory potency of a compound against the FPPS enzyme are known in the art. For example, suitable methods are described in Kavanagh K L et al, 2006, PNAS 103: 7829-7834, the contents of which, in particular, the method of assaying inhibition of FPPS described at page 7834, are hereby incorporated by reference. A method such as that described in the present Examples may be used.. In one example, a BP compound having a high inhibitory potency has an inhibitory potency equal to or greater than that of zoledronate in a particular assay. A BP compound, for example, an N-BP compound, for use in the invention may have inhibitory potency against the geranyl-geranyl pyrophosphate synthase enzyme (GGPPS) enzyme. Examples of BP compounds having such inhibitory activity include, for example, those described in US 2010/0240612 A1. Methods for determining inhibitory potency of a compound against the GGPPS enzyme are known in the art. For example, suitable methods are described in Artz J D et al, 2011, The Journal of Biological Chemistry, 286: 3315-332, the contents of which, in particular, the method of assaying inhibition of GPPS described at page 3316, are hereby incorporated by reference. A method such as that described in the present Examples may be used.

A BP compound, for example, an N-BP compound for use in the invention may have a low affinity for bone. Examples include Compound C as described herein. . Methods for determining affinity of a BP compound for bone are known in the art. Determining affinity for bone may comprise determining affinity for hydroxyapatite (HAP). Suitable methods for determining bone affinity are referred to, for example, in Ebetino et al, 2011, Bone 49: 20-33, in Table 2 page 27 (HAP FPLC, fluorescence competitive binding assay, NMR-based competitive binding assay, or constant composition kinetic studies of HAP crystal growth). The contents of Ebetino et al 2011, in particular, Table 2 and the methods referred to in the Table are hereby incorporated by reference. A method such as that described in the present Examples may be used. Without wishing to be bound by theory, it is believed that compounds having a lower affinity for bone are more easily released for inhibitory action against enzyme and may therefore have increased inhibitory effect. A BP compound may have both a high inhibitory potency on, the FPPS enzyme and a low affinity for bone. Examples include Compound C. A BP compound may have both inhibitory potency on, the GGPPS enzyme and a low affinity for bone eg Digeranyl-BP.

A BP compound, for example, an N—BP compound, for use in the invention may inhibit one or more steps in the mTOR pathway (FIG. 8). A BP compound for use in the invention may comprise one or more of the properties described herein, in any suitable combination.

A BP compound, for example, an N—BP compound, for use in the invention may comprise a compound as used herein in the Examples. Thus, such a compound may comprise any of Zoledronate, Compound A (the 1R,6S isomer of 2-Azabicyclo-[4.3.0]nonane-8,9-diphosphonic acid), Compound B (the 1S,6R isomer of 2-Azabicyclo-[4.3.0]nonane-8,9-diphosphonic acid) or Compound C (1-fluoro-2-(imidazo-[1,2-a]pyridine-3-yl)-ethyl-bisphosphonic acid). Structures of Compounds A, B and C are presented in FIG. 10.

Non-BP Compounds

In one aspect, the invention may relate to use as cytoprotectants of non-BP compounds which have BP-like activity. In particular, the invention may relate to use of compounds which have one or more properties described herein for BP compounds for use in the invention. Such properties include, for example, inhibition of one or more steps in the mevalonate pathway (e.g. inhibitory potency on the FPPS or GGPPS enzyme), low affinity for bone, or inhibition of one or more steps in the mTOR pathway, or any one or more of the cytoprotective properties described herein for a BP cytoprotectant.

In one aspect, such non-BP compounds (or pharmaceutically acceptable salts, solvates or pro-drugs thereof) may be used in the same way described herein for BP compounds.

Examples of compounds which may have BP-like properties include BP-like phosphono-phosphinate compounds, e.g. the pyridylaminomethane phosphonoalklyphosphinates described in Ebetino and Jamieson, 1990, Phosphorus, Sulfur and Silicon, 51/52: 23-26, and EP 298553. The contents of this paper, in particular, the contents describing the phosphono-phosphinate compounds and the preparation thereof, are hereby incorporated by reference.

Other examples include non-BP compound inhibitors of FPPS enzyme. For example, Jahnke et al, 2010, Nature Chemical Biology 6: 660-666 describes allosteric non-bisphosphonate inhibitors of FPPS, which may be useful in the present invention. The contents of this paper (Jahnke et al 2010, supra), in particular the contents describing the allosteric inhibitory compounds and the preparation thereof, are hereby incorporated by reference. WO 2010043584 (A1) and US 2011/288057 (A1) report inhibitors of FPPS enzyme comprising salicylic acid derivatives, which may be useful in the present invention. The inhibitors in general comprise Formula I as in each document. The contents of each of these patent applications, in particular the contents describing the inhibitory compounds and the preparation thereof, are hereby incorporated by reference. WO 2009106586 (A1) reports inhibitors of FPPS enzyme comprising arylquinoline derivatives, which may be useful in the present invention. The contents of this document, in particular the contents describing the inhibitory compounds and the preparation thereof, are hereby incorporated by reference. WO 2006072561 (A1) reports inhibitors of FPPS enzyme which may be useful in the present invention. The contents of this document, in particular the contents describing the inhibitory compounds and the preparation thereof, are hereby incorporated by reference.

Damage

Damage as used herein may refer to any suitable harmful effect. Damage may be to cells, or to a tissue, organ, or organism (subject) in which cells are located. In particular, damage may refer to a harmful effect induced by radiation and/or a chemical agent. Typically such damage occurs due to exposure of cells, tissues, organs or an organism, to radiation and/or a chemical agent, for example, any of those described herein.

In one aspect, damage to a cell as referred to herein, may comprise DNA damage in the cell. Thus, in one aspect, the invention is concerned with the cytoprotective properties of BP compounds in protecting cells against DNA damage, in particular, radiation-induced and/or chemical-induced DNA damage.

As used herein, protection of cells against DNA damage includes protection against accumulation of DNA damage in the cells. Without wishing to be bound by theory, it is believed that the BP compounds enhance DNA repair in the cells.

DNA damage may cause or contribute to damage such as, for example, reduced cell lifespan, impaired or aberrant cellular function, (premature) cell death, cell senescence, and/or aberrant cell division, which may lead to the development of cancer.

DNA Damage

In general, DNA damage as used herein refers to a harmful effect on the structure and/or function of cellular DNA, such as any of those described herein. DNA damage may be induced as a result of exposure to a DNA-damaging agent.

In general, DNA damage as referred to herein comprises damage to cellular DNA. Cellular DNA may comprise, for example, nuclear DNA, mitochondrial DNA.

The composition and structure of DNA is well known in the art. In general, undamaged DNA comprises deoxyribonucleic acid. Typically, a DNA molecule comprises a double stranded helix, where each strand in the helix comprises a polymer of units called nucleotides. Each strand generally comprises a backbone of alternating sugars (deoxyribose) and phosphate groups, with nucleobases (Guanine (G), Adenine (A), Thymine(T) or Cytosine (C)) attached to the sugars. In general there is complementary base pairing (by hydrogen bonding) between nucleobases of one strand and nucleobases on the other strand to form base pairs. Base pairs generally comprise G-C or A-T. Typically the DNA also comprises intra-strand base stacking interactions.

DNA may be supercoiled, either in the direction of the helix (positive supercoiling) or in the opposite direction (negative supecoiling). DNA may be packaged or bound to chromatin proteins, including for example, to histone protein. DNA may be located, for example, in the nucleus and/or mitochondria of a cell.

DNA structure is often referred to as including primary, secondary, tertiary and/or quaternary structure. Primary structure of DNA generally comprises the linear sequence of nucleotides (typically in a 5′ to 3′ direction) linked by phosphodiester bonds in the DNA strands. Secondary structure of

DNA generally comprises interactions between bases (which parts of which strands are bound to each other). Secondary structure typically includes, for example, base-pairing and base-stacking interactions. Tertiary structure of DNA generally comprises the three-dimensional structure, as defined by the atomic coordinates. Tertiary structure typically includes, for example, a double helical structure. Quaternary structure of DNA generally comprises a higher level organisation of DNA, for example, in chromatin or other packaging.

As used herein, DNA damage generally comprises a physical abnormality in the DNA, in particular in the DNA structure, such as any of the structures described herein.

Physical abnormality in DNA may comprise, for example, disruption to the secondary structure (e.g. disruption of the helical structure) and/or disruption to the DNA superstructure, for example, to the supercoiling, or histone packaging of the DNA. DNA damage may comprise a modification in the primary structure, for example, chemical modification of one or more bases. Such modifications may affect the secondary and/or superstructure, for example, by introducing non-native chemical bonds, or bulky adducts that do not fit the DNA helix. Damage may comprise one or more lesions in the DNA.

DNA damage may be such as to be recognised by one or more enzymes and may be repaired by one or more DNA repair mechanisms in a cell.

In one aspect, DNA damage as used herein may comprise or may cause or contribute to, a transforming or cancerous alteration in the DNA. A transforming or cancerous alteration in the DNA generally refers to an alteration which causes the cell to become cancerous. Thus, DNA damage as referred to herein may comprise an alteration in cellular DNA which causes development of a first, second or subsequent primary cancer. Such an alteration may, for example, result in aberrant cell division. In one aspect, DNA damage may comprise an alteration in DNA which causes a cell to become malignant.

DNA damage may, for example, comprise one or more of the following:

Oxidation of DNA

Oxidation of DNA, in particular of one or more bases, e.g. guanosine (e.g to form hydroxydeoxyguanosine or 8-oxo-7,8-dihydroguanine (8-oxoG). Examples of DNA-damaging agents which comprise oxidising activity include: free radicals (e.g. produced in response to UV-A light) or hydrogen peroxide.

Alkylation of DNA

Alkylation of DNA, for example of phosphotriesters, and/or of bases. Alkylation may comprise for example, methylation. Examples include 7-methylguanine, 1-methyladenine, 6-O-Methylguanine.

Intercalation Between Bases

A DNA-damaging agent may fit into a space between adjacent base pairs. Such agents are known as intercalators. In order for an intercalator to fit between base pairs, the bases must separate, distorting the DNA strands by unwinding of the double helix. This inhibits both transcription and DNA replication, causing toxicity and mutations. Typically, intercalators are aromatic and planar molecules. Examples of intercalators include ethidium bromide, acridines, daunomycin, doxorubicin and thalidomide

DNA Adduct Formation

A DNA damaging agent may cause formation of a (typically bulky) DNA adduct, which disrupts the DNA structure. In one aspect an adduct may comprise a polycyclic aromatic hydrocarbon adduct. Examples of agents which form adducts include benzo[a]pyrene diol epoxide and aflatoxin. Examples of adducts include benzo[a]pyrene diol epoxide-dG adduct and aristolactam I-dA adduct.

DNA Cross-Linking

DNA damage may comprise formation of cross-links between bases in the same or different strands, for example between adjacent bases. For example, cross-links may be formed between pyrimidine bases, e.g. thymine dimers or cytosine dimers. Examples of DNA-damaging agents which have cross-linking activity include: UV light, especially UV-B light (which causes formation of thymine dimers).

Chemical Modification of Bases

Including oxidation, alkylation, such as methylation, and formation of ethenobases.

Single or Double Stranded Breaks

Oxidation, ionising radiation, or thermal disruption for example, may cause one or more breaks of a single or double strand in the DNA.

Hydrolysis of Bases

Examples include deamination, depurination, and depyrimidination. Depurination may also be caused by thermal disruption of DNA.

Mismatch of Bases

Errors in DNA replication, in which the wrong DNA base is stitched into place in a newly forming DNA strand, or a DNA base is skipped over or mistakenly inserted.

Examples of spontaneous DNA damage include loss of a base, deamination, sugar-ring puckering and/or a tautomeric shift.

In a preferred aspect, DNA damage as referred to herein comprises one or more single or double stranded breaks in the DNA, in particular one or more double stranded breaks.

Causes of Damage and Accumulation of Damage

Damage (e.g. DNA damage) may be induced in a cell in response to one or more damaging agents. A damaging agent typically refers to any radiation, chemical substance or other factor which is able to cause damage in a cell, in particular, DNA damage. Examples of agents include radiation, and chemical agents, including any of those described herein.

A damaging agent may comprise an endogeneous agent. Typically, an endogeneous agent originates within an organism. Such an agent may be produced by a cell, tissue or organ in the organism. For example, an endogeneous agent may comprise a chemical agent generated as a by-product of cell metabolism, e.g. endogeneously formed oxygen free radicals or cellular water (which has hydrolytic activity). Endogeneous reactive oxygen species (ROS) may include, for example, superoxide, hydroxyl radicals and hydrogen peroxide.

Alternatively, a damaging agent may comprise an exogeneous agent. Typically, an exogeneous agent originates outside an organism. For example, an exogeneous agent may comprise environmental radiation, or radiotherapy, or a chemotherapeutic agent.

Damaging (e.g. DNA damaging) agents may arise in association with a disease or condition, for example, aging or an age-related disorder, physical or chemical tissue trauma, radiation-induced tissue trauma, infection, an inflammatory disorder, an autoimmune disorder, ischaemia or a condition associated with ischaemia, degenerative diseases and disorders, or chronic obstructive pulmonary disease. A damaging agent may be at least partially causative of a disease or condition and/or a disease or condition may cause production of a damaging agent.

Damage (e.g. DNA damage) induced in response to an agent typically occurs when the cell (or cellular DNA) is exposed to the agent. In the case of DNA damage, DNA may be exposed directly, or a cell(s) comprising the DNA may be exposed. Exposure of a cell(s) may be of a corresponding tissue, organ or organism containing the cell.

An agent may be tested for a damaging effect, e.g, a damaging effect on DNA, according to any suitable assay such as any of those described herein. Typically, cells (or cellular DNA), or corresponding tissue, organ or organism, are exposed to an agent under suitable conditions, and the extent of damage (e.g. DNA damage) is assayed and compared to the extent of damage in the absence of the agent. Assays for determining DNA damage are described herein.

Suitable damaging agents are known in the art, and are described further herein.

As described herein, cells generally comprise one or more repair mechanisms for repair of damaged DNA arising, for example, due to endogeneous damaging agents. However, in some instances, the rate of damage may be greater than the rate of repair, which may lead to accumulation of DNA damage in cells. This can occur, for example, where there is increased damage, e.g. due to exposure to one or more exogeneous damaging agents, or due to prolonged exposure to damaging agents as cells age and/or where there is a defect in one or more cellular repair mechanisms, e.g due to disease. A BP cytoprotectant according to the invention may be used to protect against accumulation of damage.

Damaging Agents

A damaging agent may for instance comprise radiation or a chemical agent.

Radiation Agent

A damaging radiation agent generally comprises any suitable form of radiation which is able to cause damage (e.g. DNA damage) in cells which are exposed to the radiation. Radiation, e.g. ionising radiation, may cause DNA damage, e.g. single or double-stranded breaks, as described herein.

Examples of damaging radiation include ultraviolet radiation (e.g. UVA or UVB rays, in for example, solar radiation), infrared radiation, X-rays or gamma-rays.

Radiation may occur in the environment, e.g. solar radiation. Alternatively, cells may be exposed to radiation under specific conditions, for example, during radiotherapy for the treatment of a disease or condition, e.g. cancer.

UV Light

Solar radiation (sunlight) generally includes UV radiation, for example, UV-A and/or UV-B radiation.

Exposure to UV light is often associated with cell damage (e.g. DNA-damage), in particular in skin cells, (for example keratinocytes, primary epithelial cells, basal cells, antigen-presenting cells, and skin stem cells), eye cells and immune cells.

The effects of damage, e.g. DNA damage, caused by UV exposure may be of clinical and/or cosmetic concern. DNA damage caused by UV may lead to an increase in likelihood of developing a first or subsequent primary cancer in the cells, e.g. skin cancer, including melanoma. In another example, damage may lead to increased signs of aging or other visible deterioration in cell quality in cells, for example, wrinkling of skin, thinning of skin, loss of elasticity, reduced pigmentation, fragile blood vessels, increased risk of skin injury and decreased capacity for repair following injury. BP compounds or pharmaceutically acceptable salts or solvates thereof may be used to protect against damage. (e.g. DNA damage) caused by UV light. Protection may have therapeutic and/or cosmetic benefits. For example, UV-protection of the skin may improve the health and/or appearance of the skin. Reduced damage may, for example, reduce the risk of cancer such as skin melanoma developing. Reduced damage may ameliorate one or more of the above signs of aging or other deterioration in the skin. Protection against the effects of UV light may also be useful in treating or preventing an autoimmune disease such as systemic lupus erythematosus (SLE).

A UV protectant as used herein refer to an agent which can protect cells, or a tissue, organ or organism against at least one harmful effect of UV radiation.

Radiotherapy

Radiotherapy or radiotherapeutic agent as used herein generally refers to radiation used in the treatment of a disease or condition in a subject. For example, radiotherapy is often used in treatment of cancer as described herein.

Any suitable radiation may be used. Examples include external beam radiotherapy (X ray or gamma ray). This may include proton therapy, 3-dimensional conformal radiation therapy, intensity-modulated radiation therapy, tomotherapy, image-guided radiation therapy, stereotactic radiosurgery, and/or stereotactic body radiation therapy. Other types of radiotherapy include brachytherapy (or internal radiation) and systemic radiotherapy administered orally or intravenously (e.g. radioactive iodine, or a radioactive substance bound to an antibody.)

While beneficial in treating the given disease or condition, radiotherapy often has the undesirable side-effect of causing damage (e.g. DNA damage) to healthy (e.g. non-diseased) cells which are exposed to the radiation during the treatment (the radiotherapy is indiscriminate in this respect). For example, cancer radiotherapy, intended to destroy the target cancerous cells, may also cause damage (e.g. DNA damage) to non-cancerous cells which are also exposed.

Exposure of the healthy (e,g, non-cancerous) cells typically leads to damage (e.g. DNA damage) in these cells.

DNA damage may cause, for example, increased cell death in the non-cancerous cells, which may have one or more associated side effects. Often this may limit the dose of radiotherapy which can be safely applied. For example, some radiotherapy acts by killing cells that divide rapidly, one of the main properties of most cancer cells. This means that radiotherapy also harms cells that divide rapidly under normal circumstances, for example stem cells in the bone marrow, digestive tract, and hair follicles. This results in some of the most common side-effects of radiotherapy: myelosuppression (decreased production of blood cells, hence also immunosuppression), mucositis (inflammation of the lining of the digestive tract), and alopecia (hair loss).

DNA damage in non-cancerous cells may alternatively predispose the cells to becoming cancerous, leading to a second primary cancer in the subject.

In one aspect, the present invention is particularly concerned with cytoprotection in radiotherapy. For example, the invention is particularly concerned with cytoprotection in DNA-damaging radiotherapy. A cytoprotectant for use in protecting healthy cells against damage during radiotherapy and/or chemotherapy may be referred to as a cytoprotective adjuvant.

Chemical Agents

Damage, e.g. DNA damage, may be caused by exposure to one or more chemical agents. A damaging chemical agent generally comprises any chemical substance or other factor which is able to cause damage (e.g. DNA damage) in cells which are exposed to the agent. A chemical agent may comprise a DNA-reactive chemical.

As explained herein, a damaging chemical agent may be an exogeneous or an endogeneous chemical agent.

Damaging (e.g. DNA-damaging) agents may include naturally occurring or synthetic chemical compounds or compositions. Examples include synthetic chemicals, plant toxins, dietary agents, industrial chemicals such as vinyl chloride and hydrogen peroxide, and environmental chemicals, e.g. polycyclic aromatic hydrocarbons found in smoke, soot and tar.

Examples of DNA-damaging chemicals may include: DNA reactive chemicals, (e.g. deaminating agents such as nitrous acid; polycyclic aromatic hydrocarbon (PAH), alkylating agents such as ethylnitrosourea, nitrosamines, mustard gas and vinyl chloride, aromatic amines and amides, e.g. 2-acetylaminofluorene, bromine or bromine containing compounds, sodium azide, psoralen (when combined with ultraviolet radiation), and benzene); base analogs; intercalating agents (e.g. ethidium bromide, proflavine, daunorubicin); metals (e.g. arsenic, cadmium, chromium, nickel, iron).

As above, damaging chemical agents may arise in association with a disease or condition in cells, tissue, organ, or organism. For example, inflammatory diseases may lead to increase production of nitrogen species, i.e. nitric oxide, which leads to DNA damage.

Chemotherapy

Exposure to damaging chemical agents may occur during administration of chemotherapy to a subject in need thereof. Chemotherapy or a chemotherapeutic agent as used herein generally refers to one or more chemical substances used to treat a disease or condition, for example, cancer. Often chemotherapeutic agents comprise cytotoxic antineoplastic drugs. Chemotherapy may be administered in combination with one or more other treatments, e.g. radiotherapy and/or surgery.

Damaging chemotherapeutic agents often do not discriminate between target (typically diseased) cells (e.g. target cancer cells), and other healthy (e.g. non-cancerous) cells of the host or subject which are also exposed to the agent during treatment. Such agents are generally referred to as indiscriminate chemotherapeutic agents. Exposure of the healthy (e,g, non-cancerous) cells typically leads to damage (e.g. DNA damage) in these cells.

DNA damage may cause, for example, increased cell death in the non-cancerous cells, which may have one or more associated side effects. Often this may limit the dose of chemotherapy which can be safely applied. For example, some chemotherapeutic agents act by killing cells that divide rapidly, one of the main properties of most cancer cells. This means that chemotherapy also harms cells that divide rapidly under normal circumstances, for example stem cells in the bone marrow, digestive tract, and hair follicles. This results in some of the most common side-effects of chemotherapy: myelosuppression (decreased production of blood cells, hence also immunosuppression), mucositis (inflammation of the lining of the digestive tract), and alopecia (hair loss).

DNA damage in non-cancerous cells may alternatively predispose the cells to becoming cancerous, leading to a second primary cancer in the subject.

Some newer anticancer drugs (for example, various monoclonal antibodies) are not indiscriminately cytotoxic, but rather target proteins that are abnormally expressed in cancer cells and that are essential for their growth. Such treatments are often referred to as targeted chemotherapy.

In one aspect, the present invention is particularly concerned with cytoprotection in indiscriminate chemotherapy. For example, the invention is particularly concerned with cytoprotection in DNA-damaging chemotherapy, particularly, DNA-damaging indiscriminate chemotherapy.

Certain chemotherapeutic agents also have a role in the treatment of other conditions, including ankylosing spondylitis, multiple sclerosis, Crohn's disease, psoriasis, psoriatic arthritis, systemic lupus erythematosus, rheumatoid arthritis, and scleroderma.

Chemotherapeutic agents may include, for example, one or more of the following categories of anti tumour agents:

(i) antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride; (iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras signalling inhibitors such as farnesyl transferase inhibitors, for example sorafenib (BAY 43-9006)), inhibitors of cell signalling through AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin av[33 function and angiostatin)]; and

(vi) vascular damaging agents such as Combretastatin A4 and compounds disclosed in International Patent Applications WO 99/02166, WO 00/40529, WO 00/41669, WO 01/92224, WO 02/04434 and WO 02/08213.

Assaying DNA Damage

DNA damage may be detected and/or quantified using any suitable method. Suitable methods are known in the art. For example, suitable methods include: comet assay, FLARE (fragment length analysis using repair enzymes), PCR, Tunel assay, and immunological methods (for example 8-hydroxydeoxyguanosine (8-OHdG)).

In one aspect, damaged DNA may be detected and/or determined directly. For example, double stranded breaks in DNA may be determined using the yH2AX marker. Cells may be stained for phosphorylated yH2AX, and the number of yH2AX DNA damage foci determined.

Damaged DNA may also be determined indirectly. Typically this is done by assaying another property of a cell, tissue, organ or organism which is dependent on the integrity of the DNA. For example, damage may be determined indirectly by assaying one or more effects of DNA damage as described herein.

Suitable methods are described in the present Examples.

DNA Repair

In some instances, DNA damage may be recognised by one or more enzymes and may be repaired by one or more DNA repair mechanisms in a cell. Such mechanisms are known in the art.

Applications of the Cytoprotectant

The cytoprotective properties of BP compounds find a number of applications. These include use as cytoprotective adjuvants in radiotherapy and chemotherapy, and use in the treatment of diseases or conditions associated with accumulation of DNA damage. The cytoprotectants may also be used in vitro, for example to enhance preparation of induced pluripotent stem cells.

BP Compounds as Cytoprotective Adjuvants in Radiotherapy and/or Chemotherapy

As described further herein, the inventors have provided evidence for a differential protective activity of BP compounds between cancerous and non-cancerous cells. Selectivity for non-cancerous cells allows use of BP compounds as cytoprotective agents (cytoprotective adjuvants) in cancer therapy

BP compounds may be used to protect healthy non-cancerous cells from damage (e.g. DNA damage) which might arise due to cancer radiotherapy and/or chemotherapy. Thus in one aspect, the invention is concerned with a BP compound for use as a cytoprotective adjuvant in cancer radiotherapy and/or chemotherapy.

Accordingly, the invention provides bisphosphonate (BP) compounds, or a pharmaceutically acceptable salts or solvates or pro-drugs thereof, for use in a subject as a cytoprotectant for protecting non-cancerous cells against radiation-induced damage and/or damage induced by a chemical agent, preferably wherein the subject is undergoing cancer radiotherapy and/or chemotherapy.

Also provided is use of a BP compound as described herein for the manufacture of a medicament for use as a cytoprotective adjuvant in cancer therapy. Further provided is a method of protecting non-cancerous cells in a subject from damage induced by cancer radiotherapy and/or chemotherapy comprising administering to the subject an effective amount of one or more BP compounds as described herein, in combination with the anti-cancer therapy.

An adjuvant generally comprises a substance which may be administered in combination with a given therapy (e.g. a drug or other treatment) to increase or enhance the therapeutic effect of the therapy. In the present case, protection of the non-cancerous cells may result in increased survival of these cells during and/or after cancer treatment. Protection of the non-cancerous cells may reduce one or more side effects of the cancer therapy, and/or allow an increase in dose of the cancer therapy. Side effects of cancer chemotherapy and/or radiotherapy may include, for example, myelosuppression (decreased production of blood cells, hence also immunosuppression), mucositis (inflammation of the lining of the digestive tract), alopecia (hair loss), and development of a second or subsequent primary cancer in the previously non-cancerous cells. A cytoprotective adjuvant for use to protect against damage by radiation may also be referred to as a radioprotectant.

Accordingly, a BP compound may be administered to a subject as a cytoprotective adjuvant in combination with damaging radiotherapy or chemotherapy to protect against damage in non-cancerous cells which are not a target of the therapy but which also exposed to the therapy. Suitable combination products and uses thereof are described further herein.

BP compounds may be used as cytoprotective adjuvants in the treatment of any suitable cancer, including but not limited to non-solid tumours such as leukaemia, for example acute myeloid leukaemia, multiple myeloma, haematologic malignancies (e.g. myelodysplastic syndrome or myeloproliferative syndrome) or lymphoma, and also solid tumours and their metastases such as melanoma, non-small cell lung cancer, glioma, hepatocellular (liver) carcinoma, glioblastoma, carcinoma of the thyroid, bile duct, bone, gastric, brain/CNS, head and neck, hepatic, stomach, prostate, breast, renal, testicular, ovarian, skin, cervical, lung, muscle, neuronal, oesophageal, bladder, lung, uterine, vulval, endometrial, kidney, colorectal, pancreatic, pleural/peritoneal membranes, salivary gland, and epidermoid tumours.

In general, the protective effect of the BP adjuvant is such as to reduce damage (e.g. DNA damage) in one or more non-cancerous cells, typically to an extent that is clinically detectable and/or clinically useful. The protective effect of the BP adjuvant may be such that an increased survival rate is shown in non-cancerous cells compared to that in the absence of the adjuvant. The protective effect of the BP adjuvant may be such as to reduce one or more side effect of the cancer therapy, typically to an extent that is clinically detectable and/or clinically useful. The protective effect may allow an increase in the dose of cancer therapy which can be applied.

In one aspect the invention is concerned with the use of a BP compound as a cytoprotective adjuvant to reduce the occurrence or extent of at least one side effect of cancer radiotherapy and/or chemotherapy.

Typically, the protective effect of a BP compound is selective for non-cancerous cells compared to cancerous cells such that the above beneficial effects in non-cancerous cells are achieved without significantly decreasing the effectiveness of the cancer treatment in the target cancerous cells. In one aspect, the cancer therapy is unaffected to an extent that it is clinically useful. Preferably there is no detectable decrease in effectiveness of the cancer therapy. The effectiveness of the cancer therapy may be assessed by conventional means such as the response rate, the time to disease progression and/or the survival rate. Effectiveness of the cancer therapy may for example, be assessed in terms of anti-tumour effects including but not limited to, inhibition of tumour growth, tumour growth delay, regression of tumour, shrinkage of tumour, increased time to regrowth of tumour on cessation of treatment, slowing of disease progression.

BP Compounds as Cytoprotectants Against Solar Radiation

Solar radiation (e.g. UV radiation) is a potential cause of cellular damage, including DNA damage. BP compounds may be used as cytoprotectants to protect cells against damage (e.g. DNA damage) induced by solar radiation.

Cells which are particularly vulnerable to damage by solar radiation include skin cells (for example fibroblast, primary epithelial cells, basal cells, antigen-presenting cells, and skin stem cells), eye cells and immune cells.

Exposure to solar radiation, and associated damage to DNA, can result in increased transformation of non-cancerous cells to cancerous cells, with development of a primary cancer, e.g. a skin melanoma. Exposure to intense radiation (e.g. strong sunlight) can result in burning, e.g. of skin cells.

As described further herein, radiation-induced damage to skin cells, e.g. fibroblasts or skin stem cells, can result in (premature or increased) aging of the skin. For example, damage to stem cells can result in impaired tissue regeneration by the stem cells. Signs of skin aging include: wrinkling of skin or other deterioration in the appearance of skin, for example, thinning of skin, loss of elasticity, reduced pigmentation, fragile blood vessels, increased risk of skin injury and decreased capacity for repair following injury.

Protection against solar radiation-induced damage by BP compounds may have therapeutic benefit, for example, reduced risk of cancer development, increased capacity for repair after injury. Protection may also provide non-therapeutic, e.g. cosmetic benefit, e.g. reduced wrinkling, or reduced scarring.

Thus the invention is concerned with use of BP compounds as cytoprotective skin care agents for therapeutic or non-therapeutic purposes. The BP compounds may be used as cytoprotective agents in sunscreen formulation, or in skin care compositions, e.g. anti-aging compositions.

Protection Against Development of Primary Cancers

As described herein, protection against DNA damage in cells may protect the cells against a transforming mutation in the DNA. This may protect the cells against development into a first or subsequent primary cancer.

Promotion of Tissue Regeneration by Protected Stem Cells

BPs may be used to protect stem cells against damage, e.g. DNA damage. It is believed that DNA damage in stem cells contributes at least in part to loss of stem cell function (e.g. proliferative capacity, and/or differentiation ability), and therefore a limited stem cell lifespan.

Stem cells (and particularly stem cell functions) are needed for tissue maintenance and repair, which can be more generally described as tissue regeneration.

In a healthy situation stem cells divide throughout the life cycle of an organism to maintain the stem cell pool while some undergo differentiation to replace the mature cell types which comprise the tissue. This may be referred to as tissue maintenance. When stem cells are unable to maintain this balance due to decreased proliferation capacity and/or differentiation ability, such as after DNA damage, there is loss of tissue maintenance. In this case the tissue needs to be regenerated through a boost of stem cell activity.

In situations of tissue injury or disease, stem cells will divide and produce sufficient number of mature cells to regenerate the injured tissue, still maintaining some undifferentiated stem cells in the pool to guarantee tissue maintenance with time. This may be referred to as tissue repair. Tissue repair can occur by stimulation of endogenous stem cells to proliferate and differentiate. For serious tissue damage, e.g. high dose chemotherapy or radiotherapy, or in cases where stem cells are defective (e.g. due to inherited disorders), stem cells can be manipulated in vitro and transplanted into a subject to repair the tissue.

Both tissue maintenance and repair comprise proliferation and differentiation of stem cells. In particular, both maintenance and repair as used herein comprise proliferation of stem cells to regenerate the stem cell pool or compartment. Tissue maintenance or repair by stem cells is therefore self-renewing or self-sustaining as regards the stem cells. This sustainable maintenance and repair is distinct from, for example, mere acceleration of differentiation of stem cells without regeneration of stem cells themselves (which does not maintain the stem cell pool).

Tissue regeneration as used herein generally refers to regeneration of any tissue type towards a healthy state. This includes both regeneration of functionality (e.g. of stem cell functionality, for example of a bone marrow compartment) and regeneration of structure and architecture associated with function of a tissue (e.g. skin epidermal structure).

A BP cytoprotectant according to the invention, which may be used to protect stem cells against DNA damage, may be used to promote tissue regeneration by stem cells, in vivo or in vitro.

Promotion of tissue regeneration as used herein refers to the ability of a BP cytoprotectant to increase tissue regeneration by at least a detectable amount compared to regeneration in the absence of the BP cytoprotectant.

A BP cytoprotectant may be used to promote tissue regeneration in vivo, generally for tissue maintenance and/or repair, in a subject in need thereof,. Tissue regeneration may be needed in a subject, for example, to treat tissue damage.

As used herein, treatment may be therapeutic or prophylactic. Treatment may also comprise cosmetic treatment. Accordingly, treatment of tissue damage may be for therapeutic or cosmetic purposes. Treatment may be prophylactic, e.g. to maintain tissue and prevent or reduce future tissue damage. Treatment may be therapeutic, e.g. to repair or restore damage which has already occurred.

Tissue damage as used herein generally refers to any harmful effect in a tissue and/or the cells of a tissue. Damage may be structural and/or functional. For example, damage may comprise loss of one or more cells in the tissue. Loss of cells (and tissue damage) may be due, for example, to natural cell death or to pathological cell death, e.g. due to disease or injury. Damage may comprise reduction or loss of one or more cell or tissue functions. As used herein, tissue damage encompasses tissue destruction and/or loss of tissue.

Tissue damage may occur in a subject or organism in association with a number of diseases and conditions. Such diseases or conditions may for example, be selected from: physical or chemical tissue trauma, radiation-induced tissue trauma, ischaemia or conditions associated with ischaemia, aging or an age-related disorder, inflammatory disorders, degenerative diseases or disorders, stem cell diseases or disorders, chronic obstructive pulmonary disease, infections and autoimmune disorders.

BP compounds may be used to treat any disease or condition in which tissue regeneration is beneficial, including the diseases or conditions above. Treatment may be therapeutic or cosmetic. BP compounds may therefore find use in regenerative or cell based therapeutics and/or in cosmetic treatment.

Promotion of tissue regeneration may be useful in agriculture or the food industry, e.g. in fish farming.

Promotion of tissue regeneration may be useful in vitro in culture of stem cells, e.g. for use in stem cell transplantation techniques.

Treatment of Diseases or Conditions Associated with Accumulation of DNA Damage

As described herein, BP cytoprotectants may be used to treat diseases or conditions which are associated with accumulation of DNA damage. Such diseases or conditions may be caused by or may cause accumulation of DNA damage in cells, for example, due to increased DNA damage in the cells (e.g. because of increased exposure to a DNA damaging agent), and/or due to defective DNA repair in cells.

Examples of diseases or conditions include: physical or chemical tissue trauma, radiation-induced tissue trauma, ischaemia or conditions associated with ischaemia, aging or an age-related disorder, inflammatory disorders, degenerative diseases or disorders, stem cell diseases or disorders, chronic obstructive pulmonary disease, infections and autoimmune disorders.

Physical or Chemical or Radiation-Induced Tissue Trauma

Examples of physical or chemical tissue trauma include: wounding, cancer chemotherapy, thermal damage, water damage, damage due to exposure of cells to naturally occurring or synthetic chemicals. Damage may occur as a result of radiation-induced tissue trauma, for example, damage due to cancer radiotherapy, solar radiation (e.g. UV radiation), infrared, X-rays or gamma-rays.

Damaging chemical agents and radiation are described elsewhere herein. Wounding or physical injury may be of any suitable tissue, for example skin tissue, or gut mucosa. Promotion of wound healing may be therapeutic or cosmetic.

Ischaemia and Conditions Associated with Ischaemia

Ischaemic damage generally occurs due to a restriction in blood supply to tissue. Inadequate blood supply, (and ischaemia) may be associated with a number of diseases or conditions, for example: atherosclerosis, ischaemic heart disease, tachycardia, hypoglycaemia, hypotension, thromboembolism, sickle cell disease, frostbite, peripheral artery occlusive disease, blood vessel rupture or anaemia. Ischaemic damage may occur in any suitable tissue or organ, for example, cardiac tissue (ischaemic heart disease), bowel tissue (e.g. ischaemic colitis, mesenteric ischaemia), brain tissue (e.g ischaemic stroke) or limb tissue.

Without wishing to be bound by theory,it is believed that ROS-induced damage in cells (e.g. DNA damage) may be associated with cardiac failure. In one aspect, a BP cytoprotectant may be used to treat cardiac failure.

Aging and Age-Related Disorders

DNA damage may occur in association with aging or an age-related disorder. For example, cells tend to accumulate DNA damage over time. Aging of some cells, e.g. skin cells, can also be accelerated by exposure to damaging-agents, such as solar radiation, as described herein. Aging of stem cells (in vivo and in vitro) generally leads to reduction and loss of one or more stem cell functions (proliferation capacity and/or differentiation ability), and is believed to be caused at least in part by accumulation of DNA damage. This generally leads to reduction in tissue regeneration ability. This is a particular problem for stem cells which perform maintenance regeneration in a subject (e.g. skin stem cells, epithelial stem cells, or hematopoietic stem cells). Reduced tissue regeneration by aging stem cells may cause one or more signs of aging in a tissue. Some tissues show one or more visible signs of aging. For example, aging in skin cells may lead to wrinkling of skin, thinning of skin, loss of elasticity, reduced pigmentation, fragile blood vessels, increased risk of skin injury and decreased capacity for repair following injury. By protecting cells against DNA damage, a BP cytoprotectant may be used to treat one or more signs (optionally visible) of aging, for example, in skin. Treatment may be therapeutic or cosmetic. For example, therapeutic benefits of treatment of aging may include reduced risk of cancer development, or increased capacity for repair after injury. Non-therapeutic benefits may include, for example reduced wrinkling, more even pigmentation or increased elasticity.

DNA damage may be associated with age-related disorders. These include, for example, sarcopenia, chronic obstructive pulmonary disorders, Alzheimer disease.

Inflammatory Disorders

DNA damage may occur in association with an inflammatory disorder. These disorders are typically associated with chronic inflammation and/or inflammatory abnormalities. Examples include inflammatory bowel disease (IBD), colitis, inflammatory arthritis (eg rheumatoid arthritis, osteoarthritis), bursitis, cystitis, dermatitis, phlebitis, rhinitis, tendonitis, tonsillitis, vasculitis, acne, asthma, autoimmune diseases, chronic prostatitis, glomerulonephritis, hypersensitivities, pelvic inflammatory disease, reperfusion injury, sarcoidosis, transplant rejection and inflammatory myopathies.

Stem Cell Diseases and Disorders

DNA damage may be associated with a defect in stem cells, e.g. due to disease or disorder. This may occur, for example, in stem cells which are particularly susceptible to accumulation of DNA damage, e.g. cells which have a defect in a DNA repair mechanism. For example, Fanconi anaemia is associated with a defect in a DNA repair mechanism in cells, in particularly in haematopoietic stem cells. The defect leads to reduced stem cell function (e.g. tissue regeneration), and consequent tissue damage - in particular an inability to produce blood cells.

Degenerative Diseases and COPD

DNA damage may be associated with other diseases or conditions, including: degenerative disease or conditions, for example Alzheimer's disease; chronic obstructive pulmonary disease (COPD), for example chronic bronchitis or emphysema.

Infections

DNA damage may occur as a result of infection - for example, bacterial infection including tuberculosis, viral infection, or fungal infection.

Autoimmune Disorders

DNA damage may occur in association with an autoimmune disorder. Examples include Addison's disease, coeliac disease, dermatomyositis, Graves disease, Hashimoto's thyroiditis, multiple sclerosis, myasthenia gravis, pernicious anaemia, reactive arthritis, rheumatoid arthritis, Sjogren syndrome and systemic lupus erythematosus.

A BP cytoprotectant may be used to treat one or more of the diseases or conditions described herein.

Induced Pluripotent Stem Cell Preparation

Induced pluripotent stem cells are generally derived from multipotent cells or somatic cells, e.g. skin fibroblasts. To prepare the induced pluripotent stem cells, the multipotent or somatic cells are genetically reprogrammed to be pluripotent. Methods for reprogramming the cells are known in the art (see, for example, Cell, 2007, 131 (5) 861-872).

Accumulation of DNA damage in the multipotent or somatic cells can reduce the efficiency of reprogramming. BP cytoprotectants may be used to protect the multipotent or somatic cells against such DNA damage and may therefore enhance preparation of induced pluripotent stem cells.

Thus, in one aspect the invention relates to a method of preparing induced pluripotent stem cells, the method comprising:

(a) administering an effective amount of a bisphosphonate (BP) compound, or a pharmaceutically acceptable salt or solvate or pro-drug thereof, to one or more multipotent or somatic cells; and

(b) preparing induced pluripotent stem cells from the multipotent or somatic cells.

Stem Cell Transplantation and Gene Therapy

A BP compound may be used as a cytoprotectant in stem cell transplantation or gene therapy techniques.

In some instances, regeneration to treat damaged tissue in a subject comprises transplantation of cells into the subject (the recipient). In general transplantation techniques are used to treat severe tissue damage. For example, stem cell transplantation may be used to treat cancer patients (e.g. leukaemia, lymphoma or myeloma patients) who are receiving doses of chemotherapy and/or radiotherapy sufficient to damage (typically destroy) stem cells in the patient (e.g. some or all of the bone marrow stem cells). Stem cells (e.g. bone marrow stem cells) may be transplanted into the patient to regenerate the stem cells (e.g. to regenerate the bone marrow compartment). Stem cell transplantation may also be used in the treatment of other conditions, for example, retinitis pigmentosa (RP) and age-related macular degeneration (AMD), cardiac diseases, autoimmune diseases. musculoskeletal and joint diseases, neurological diseases.

In general, stem cell transplantation for tissue regeneration comprises: harvesting stem cells from a suitable source; culturing the stem cells in vitro; and transplanting the cultured cells into the recipient subject.

Stem cells may be harvested, for example, from the recipient subject themselves (an autologous transplant), from a suitable donor subject (an allogeneic transplant) or from umbilical cord. Often cells are harvested from bone marrow or blood.

In vitro culture typically comprises expanding the stem cell population to obtain an (therapeutically or cosmetically) effective number and quality of stem cells, and optionally treating the cells to initiate at least some differentiation into a desired tissue type. The expanded and optionally (partially) differentiated cells are then transplanted into the recipient.

Gene therapy typically comprises: harvesting stem cells from a suitable source; manipulating the cells to transfer DNA (e.g. one or more genes) of interest into the cells; culturing the stem cells in vitro; and transplanting the cultured cells into a recipient subject.

Stem cells may be harvested, for example, from the recipient subject themselves (an autologous transplant), from a suitable donor subject (an allogeneic transplant) or from umbilical cord. For example, cells may be harvested from bone marrow or blood, or other tissue sources

In vitro culture typically comprises expanding the stem cell population to obtain an (therapeutically or cosmetically) effective number and quality of stem cells. The expanded cells are then transplanted into the recipient.

A difficulty with stem cell transplantation and gene therapy is in obtaining sufficient numbers of functional stem cells for the technique to be effective. As described herein, stem cells tend to lose some or all of their function (e.g. proliferative capacity and/or differentiation ability) with age (i.e. over time in culture). This loss of function is believed to be due, at least in part to accumulation of DNA damage. Often the donor subject (or recipient, if also the source of the stem cells) is treated with drugs (e.g. growth factors) before stem cells are harvested to increase cell numbers. The recipient may also be treated with drugs (e.g. growth factors) after transplant to increase cell numbers.

A BP cytoprotectant according to the second aspect of the invention may be administered at any stage of the procedure (e.g. to the donor before harvesting, to a recipient before harvesting or after transplant, or to the cells in vitro) to protect the cells against DNA damage, and thus improve the efficiency of the procedure. A BP cytoprotectant may be administered in combination with, e.g. one or more growth factors, as described herein.

Protective Properties of the BP Cytoprotectant

A BP cytoprotectant according to the invention generally protects one or more cells against damage (e.g. DNA damage), and in particular, damage induced by radiation and/or a chemical agent. A BP cytoprotectant may protect a tissue, organ or organism within which the cells occur.

Reference to protection against damage or reduction of damage as used herein may refer to reduction in average damage in a population of cells, e.g. in a cell culture.

A BP cytoprotectant may protect one or more cells against the effect of one or more damaging agent, including any of those described herein. Protection against damage as used herein may comprise reducing the extent of (one or more types of) damage in a cell.

In general, a BP cytoprotectant protects one or more cells against damage to at least a detectable extent according to any suitable assay for damage. A BP cytoprotectant may, for example, reduce damage by at least 5, 10, 20, 30, 40, 50, 60, 70, 80, 90 or 100% compared to that in the absence of the BP cytoprotectant.

A BP cytoprotectant may protect cells against DNA damage. Protection of cells against DNA damage as used herein may refer to protection against accumulation of DNA damage in cells. It is to be understood therefore that reference to, for example, reducing DNA damage, or to effects of DNA damage, may refer to reducing accumulation of DNA damage or to effects of accumulation of DNA damage.

Protection by a BP compound may comprise enhancing or promoting DNA repair, (for example by enhancing one or more DNA repair mechanisms), so that the extent of DNA damage remaining in the cell after occurrence of DNA damage is reduced. For example, a BP cytoprotectant may increase the efficiency of one or more repair mechanisms. A BP cytoprotectant may increase the rate of detection of, and/or repair of, one or more types of DNA damage. Without wishing to be bound by theory, it is believed that BP compound inhibition of the mTOR pathway (FIG. 8) may leads to translocation of foxo3a to the nucleus of the cell and to an increase in autophosphorylation of ataxia telangiectasia mutated (ATM) which initiates a DNA repair response.

A BP cytoprotectant may protect against damage which is an effect of DNA damage. In general, such damage is secondary to DNA damage and is associated with or caused by the DNA damage. DNA damage may contribute to the damage. The precise nature of the damage may depend upon the type of cell in which the damage occurs, and in that sense may be cell-specific. A BP cytoprotectant may protect one or more cells, (or a corresponding tissue, organ or organism comprising the cells) against one or more effect of DNA damage.

Effects of an accumulation of DNA damage may include for example, reduced life span, increased rate of cell aging, cell senescence, increased rate of cell death (apoptosis), impaired or aberrant cell function or loss of cell function, aberrant cell division, or increased probability of developing a primary cancer in a cell.DNA damage in stem cells may for example, contribute to reduction and loss of proliferative capacity and/or differentiation ability, and to impaired regenerative capacity. Other examples of effects of DNA damage include: increased death rate in non-cancerous cells (e.g. stem cells such as bone marrow stem cells) exposed to DNA-damaging cancer therapies; increased probability of a primary cancer in previously non-cancerous cells; aging of cells, e.g. epithelial or skin cells; increased rate of senescence in cells, e.g. stem cells, including hMSCs; reduced clonogenic ability in stem cells, e.g. hMSCs; reduced regenerative ability in stem cells, e.g. blastema cells, for example. of the zebrafish caudal fin.

A BP cytoprotectant may reduce DNA damage, or one or more effects of DNA damage, by at least 5, 10, 20, 30, 40, 50, 60, 70, 80, 90 or 100% in a suitable assay, as compared to damage in the absence of the cytoprotectant. Suitable assays for DNA damage are known in the art and are described herein. A BP cytoprotectant may show a protective effect as described herein in any one or more of these assays.

Protective effect of a cytoprotectant may be assessed at a suitable time, for example, following exposure to a damaging agent.

In one example, an assay for ability of a compound to reduce DNA damage may comprise exposing cells to irradiation in the presence and absence of the compound; staining the cells with a suitable marker for DNA damage (e.g. yH2AX); and comparing the amount of DNA damage (e.g. the number of DNA damage foci per cell) in the presence and absence of the compound.

A BP cytoprotectant typically protects cells to a suitable degree in the circumstances, for example for the particular cells or subject, and purpose or objective of use. For example, protection may be such that there is detectable benefit to the cells, tissue, organ or subject.

For example, a BP cytoprotectant may protect cells against damage (e.g. DNA damage) to an extent that is clinically (e,g. therapeutically) or otherwise (e.g. cosmetically) effective in the context.

Clinically (or therapeutically) effective protection against damage (e.g. DNA damage) may be considered to occur, for example, if there is a detectable improvement in the clinical condition of the subject. There may be, for example, a detectable improvement in one or more presenting symptoms of a disease or condition. There may be a detectable improvement in response of a subject to a given therapy, for example, a detectable reduction in one or more side effect of a therapy. There may be an improvement in prognosis for the subject. Methods for assessing clinical condition, symptoms, responses and prognosis in subjects are known in the art.

Cosmetically effective protection against damage (e.g. DNA damage) may be considered to occur, for example, if there is a detectable improvement in the cosmetic condition of the subject. There may be a detectable improvement in one or more signs or indicators of the cosmetic condition. Methods for assessing cosmetic condition are known in the art.

Therapeutic and cosmetic benefits of a cytoprotectant according to the invention are described herein.

A BP cytoprotectant according to the invention may be used as an adjuvant (a cytoprotective adjuvant) in radiotherapy and/or chemotherapy, e.g. in cancer radiotherapy and or cancer chemotherapy. Typically, a cytoprotective adjuvant protects a subject against one or more harmful effects of the radiotherapy and/or chemotherapy. In particular, an adjuvant may reduce one or more side effects of the radiotherapy and/or chemotherapy.

For example, a cytoprotective adjuvant typically protects non-cancerous cells exposed to damaging (e.g. DNA-damaging) radiotherapy and/or chemotherapy. In general, the protective effect of the BP adjuvant is such as to reduce damage (e.g. DNA damage) in one or more non-cancerous cells, typically to an extent that is clinically detectable and/or clinically useful. The protective effect of the BP adjuvant may be such that an increased survival rate is shown in non-cancerous cells compared to that in the absence of the adjuvant. The protective effect of the BP adjuvant may be such as to reduce one or more side effects of the therapy, typically to an extent that is clinically detectable and/or clinically useful. The protective effect may increase tolerance to the therapy, and may allow an increase in the dose of therapy which can be applied. A cytoprotective adjuvant may reduce the frequency of development of cancerous mutations in the non-cancerous cells, so reducing the likelihood of development of a (second) primary cancer in these cells.

In a further example, a BP cytoprotectant may be used to protect a subject, or cells of a subject, against damage, e.g. DNA damage, associated with solar radiation (e.g. UV radiation). Such a cytoprotectant may, for example, protect a subject to an extent that there is detectable therapeutic and/or cosmetic benefit in the subject - or in cells or a tissue of the subject, e.g. in any of skin cells, eye cells or immune cells.

As used herein, an increase or decrease or improvement in a particular property in response to a BP cytoprotectant is generally as detectable within the limits of the given assay or test.

An increase or decrease or improvement in a particular property caused by a BP cytoprotectant may comprise a statistically significant increase or decrease or improvement. Methods for determining statistical significance are known to those in the art. In one aspect, the degree of significance is such as to render in a BP cytoprotectant suitable for the intended use, e.g. clinical and/or cosmetic use.

A protective effect of a BP cytoprotectant, including any of those described herein, may be exhibited at a particular amount (e.g. dose) or concentration (an effective or protective amount, dose or concentration). Such a dose may comprise for example, a dose suitable for clinical or cosmetic use.

Selectivity for Non-Cancerous Cells

A BP cytoprotectant according to the invention typically exhibits differential protective activity between cancerous cells and non-cancerous cells. In general a cytoprotectant has protective activity which is reduced, or absent, in cancerous cells compared to protective activity in non-cancerous cells. In one aspect, a cytoprotectant exhibits no detectable protective effect in cancer cells in a given assay.

Cancer cells in which a BP compound exhibits reduced or absent protective activity may comprise cells of any suitable cancer, including any of those described herein. In one aspect, the cancer cells may comprise bone cancer cells, breast cancer cells, prostate cancer cells, and/or multiple myeloma cancer cells, leukaemia, or colon cancer. In one aspect the cancer cells may comprise 5T33 multiple myeloma cells or osteosarcoma cells such as osteosarcoma MG63 cells.

A non-cancerous cell may comprise a non-transformed cell. Typically such a cell does not comprise a transforming (or cancerous) mutation. A transforming mutation is generally one which is associated with or causes cancer in a cell, or which predispose a cell to cancer. A non-cancerous cell typically does not exhibit and/or is not predisposed to aberrant (increased) cell division. In one aspect, a non-cancerous cell may comprise a non-malignant cell.

Tests for determining cancerous and malignant cells are known in the art (see for example, Harrison's Principles of internal Medicine 18th edition, (Longo, Fauci, Kasper, Hauser, Jameson & Loscalzo).

Selective activity of the BP compounds as between cancerous cells and non-cancerous cells may be exhibited at a particular amount (e.g. dose) or concentration of BP compound, for example, at a therapeutically effective amount or dose.

Selective protective activity may be identified by determining a protective effect of a BP compound in non-cancerous cells and in cancerous cells according to any one or more of the methods described herein, and comparing the activities.

In one aspect, the difference in protective activity in cancerous v (non-cancerous) cells in a given assay is statistically significant. Methods for determining statistical significance are known to those in the art. In one aspect, the degree of significance is such as to render a BP compound suitable for clinical and/or cosmetic use

Typically, a BP compound is selective to a suitable extent in the circumstances, for example for the particular cells or subject, cancer, or cancer treatment agent.

In one example, the protective effect in cancerous cells, as determined in a given assay (for example, any of the assay methods described herein), is reduced by at least 10, 20, 30, 40, 50, 60, 70, 80, 90 or 100% compared to that determined in non-cancerous cells in the same assay. In one aspect, there is no detectable protective effect in cancerous cells in a given assay.

In one example, the degree of selectivity is such that the BP compound may be used as a cytoprotective adjuvant in cancer therapy. As described herein, such an adjuvant typically protects healthy (non-cancerous) cells exposed to damaging cancer therapy to an extent that is clinically detectable and/or clinically useful. In general, the protective activity of the BP cytoprotectant is selective for the non-cancerous cells compared to the cancer cells to an extent that there is a clinically detectable and/or clinically useful effect in the non-cancerous cells or subject, without significantly decreasing the effectiveness of the cancer therapy. In one aspect, the cancer therapy is unaffected to an extent that it is clinically useful. Preferably there is no detectable decrease in effectiveness of the cancer therapy. For example there may be a clinically detectable or clinically useful reduction in at least one side effect of the cancer therapy, without significantly decreasing the effectiveness of the cancer therapy. In another example, there may be a clinically detectable or clinically useful increase in tolerance to cancer therapy. This may, for example, allow an increase in dose without a clinically significant increase in side effects. Effectiveness of the cancer therapy may be assessed by conventional means such as the response rate, the time to disease progression and/or the survival rate

Target Cells

Cells to be protected according to the invention may comprise any suitable cells, from any suitable species, e.g. animal or human, for example, mammalian, as described herein. Typically the cells comprise non-cancerous cells or non-malignant cells as described herein.

Cells may comprise somatic cells, or stem cells.

Somatic Cells

Any suitable somatic cells may be treated. Target cells are selected according to the particular application.

For example, in use to protect against damage by solar radiation, target somatic cells may comprise skin cells (e.g. fibroblast, primary epithelial cells, basal cells, antigen-presenting cells, and skin stem cells), eye cells and immune cells.

For example, in use to protect against damage by radiotherapy and/or chemotherapy, target somatic cells may comprise any somatic non-cancerous cells which are also exposed to the radiation or chemical agent, e.g cells of the intestinal tract which may be damaged by radiotherapy directed towards the pelvis.

Stem Cells

In one aspect, a BP cyotprotectant according to the invention is for use to protect stem cells.

Any suitable stem cells may be treated including for example, embryonic stem cells, adult stem cells, stem cells derived from umbilical cord, or induced pluripotent stem cells. Target stem cells may be selected according to the particular application.

Stem cells may be pluripotent (e.g. embryonic, or induced pluripotent stem cells). Stem cells may be mulitpotent. Most adult stem cells are believed to be multipotent.

Suitably, the stem cells for protection comprise adult stem cells or induced pluripotent stem cells including any of those described herein.

Adult stem cells have been identified in many organs and tissues, including brain, bone marrow, peripheral blood, blood vessels, skeletal muscle, skin, teeth, heart, gut, liver, ovarian epithelium, adipose tissue and testis.

Specific examples of adult stem cells include: hematopoietic stem cells, mesenchymal stem cells (or bone marrow stromal cells), epithelial stem cells, brain stem cells, and skin stem cells.

Hematopoietic stem cells are found in blood and typically may differentiate to provide any of the blood cell types.

Mesenchymal stem cells are found in, e.g. bone marrow, adipose tissue or muscle, and typically may differentiate to provide any of bone cells (preosteoblasts, osteoblasts and osteocytes), cartilage (chondrocytes), fat cells (adipocytes), cells that support the formation of blood cells, and fibrous connective tissue.

Brain stem cells typically may differentiate to provide any of astrocytes, oligodendrocytes, and neurons.

Epithelial stem cells are found in the lining of digestive tract and typically may differentiate to provide any of absorptive cells, goblet cells, paneth cells and enteroendocrine cells.

Skin stem cells include epidermal stem cells (found in the basal layer of the epidermis) and follicular stem cells (found at the base of hair follicles). Epidermal stem cells generally may differentiate to provide keratinocytes. Folicular stem cells generally may differentiate to provide any of hair follicle cells and epidermal cells.

Stem cells may comprise any of those used in the present Examples, e.g. (human) mesenchymal stem cells, in particular, bone-marrow derived mesenchymal stem cells.

Induced pluripotent stem cells are derived from multipotent cells or somatic cells, which have been reprogrammed to be pluripotent.

Target Cells Further Described

A BP compound may be used to treat cells prophylactically or therapeutically. Cells, tissues or organisms to be targeted are typically those in which it is known or suspected, that damage (e.g. DNA damage) has occurred, is occurring or will occur. A BP compound may be used before, during or after damage has occurred.

Target cells may have been, or be being, exposed to, or be at risk of exposure to, a damaging (e.g. DNA-damaging) agent, including any of those described herein. In one instance, the cells have been, are being, or will be, exposed to damaging radiation and/or one or more damaging chemical agent. For example, target cells may comprise healthy (e.g. non-cancerous) cells, in a subject also having diseased, e.g. cancerous cells, wherein, when the diseased cells are treated with a damaging therapy, the healthy cells are also exposed to the damaging therapy. Target cells may comprise skin cells, e.g. skin stem cells, in a subject that is to be exposed to potentially damaging UV-rays, e.g. in strong sunlight.

A BP compound as described herein, may be applied or administered before, during or after, exposure of cells, tissue or organism to a damaging (e.g. DNA-damaging) agent, including any of those described herein. A BP compound may be applied before, during or after damaging cancer treatment as described herein.

In use to treat diseases or conditions which are associated with accumulation of DNA damage, such as any of those described herein, target cells may, for example, comprise somatic cells or stem cells of the diseased or e.g. injured, tissue.

In one aspect, target cells as described herein do not comprise bone cells. Bone cells may, for example, comprise any one or more of pre-osteoblasts, osteocytes, osteoblasts or osteoclasts Additionally or alternatively, in one aspect, target cells as described herein do not comprise mesenchymal stem cells, in particular, mesenchymal stem cells which will differentiate to produce bone cells. In one aspect, a target tissue herein does not comprise bone tissue.

Subjects

The invention may be practised in any suitable organism or subject. A subject typically comprises target cells as described herein. In one aspect, the subject is an animal or a human, for example, a mammal. A subject may be a cancer patient.

In one aspect, the present methods may additionally comprise selecting a subject in need of treatment, and administering to the subject an effective (e.g. a therapeutically or cosmetically effective) dose of a BP compound or pharmaceutically acceptable salt or solvate thereof, as described herein.

Combination Methods and Products

Combination Methods

In one aspect of the invention, BP compounds (or pharmaceutically acceptable salts or solvates thereof) may be used in combination with each other, or with other active agents.

Thus the methods and uses described herein may comprise use of one or more BP compounds or pharmaceutically acceptable salts or solvates thereof as described herein.

The one or more BP compounds or pharmaceutically acceptable salts or solvates thereof may be applied as a sole treatment (i.e. as the only active agent(s)). Alternatively, the one or more BP compounds or pharmaceutically acceptable salts or solvates thereof may be applied in combination with one or more other active agents.

Any suitable active agent may be used. In one aspect the activity of an agent used in combination with a BP compound is complementary to that of the BP compound.

In one example, a BP compound may be used as a cytoprotectant adjuvant in combination with one or more damaging treatment agents (for example, a radiotherapeutic agent and/or a chemotherapeutic agent, e.g. a cancer radiotherapeutic agent and/or a cancer chemotherapeutic agent). Additionally one or more further active agents may be combined in the cancer therapy, for example, another cytoprotectant, e.g. a radioprotectant such as Amifostine.

An active agent for use in combination with a BP compound may comprise a substance (e.g. chemical compound) or other factor which also protects against damage (e.g. DNA damage). Such an active agent may protect by a different mechanism to the BP compound. In one example, where a BP compound is to be used to protect against damage induced by radiation or a chemical agent, the BP compound may be used in combination with an active agent comprising a drug or other factor which also protects against damage induced by the same radiation or chemical agent, e.g. UV radiation. Where a BP compound is used in the treatment of a disease or condition, the BP compound may be used in combination with an active agent comprising a drug or other factor for treatment of the same disease or condition.

A BP compound or pharmaceutically acceptable salt or solvate thereof may, for example, be applied in combination with one or more active agents selected from: damaging cancer treatment agents (e.g. cancer radiotherapeutic agents and/or cancer chemotherapeutic agents), cytoprotective agents or cytoprotective adjuvants, inhibitors of the mevalonate pathway, inhibitors of mTOR signalling, anti-inflammatory agents, immunomodulatory agents, UV-protectants, anti-infectives, and cardiac medications for heart disease and cardiovascular conditions. Damaging cancer therapeutic agents are described elsewhere herein.

Cytoprotective agents or adjuvants include, for example, antioxidants, e.g. Amifostine (Koukourakis M I, Am J Clin Oncol 2012, May 24, “Dose Escalation of Amifostine for Radioprotection During Pelvic Accelerated Radiotherapy”; Gomez H L, Hematol Oncol Stem Cell Ther 2012; 5(3):152-7 “Addition of amifostine to the CHOP regimen in elderly patients with aggressive-non Hodgkin lymphoma: a phase II trial showing reduction in toxicity without altering long-term survival”; Duval M, Daniel S J, J Otolaryngol Head Neck Surg 2012 Oct. 1; 41(5):309-15 “Meta-analysis of the Efficacy of Amifostine in the Prevention of Cisplatin Ototoxicity”.) . Amifostine is clinically approved as a radioprotectant, and may protect healthy tissues from chemotherapy too. Without wishing to be bound by theory, it is believed that Amifostine acts by a different mechanism than the present BP compounds (it is a scavenger of oxygen radicals and reduces formation or oxygen radicals, thus preventing DNA damage). Accordingly it is believed that the protective activities of Amifostine and the present BP compounds are complementary.

Inhibitors of the mevalonate pathway (FIG. 3A) may act at any stage of the pathway. For example, inhibitors include statins. Statins are believed to inhibit hydroxymethylglutaryl CoA reductase, and reduce cholesterol biosynthesis. Inhibitors may for example, act to inhibit farnesyl pyrophosphate synthase (FPPS) or geranylgeranylpyrophosphate synthase (GGPPS).

Inhibitors of mTOR signalling (FIG. 8A) may act at any stage of the mTOR pathway. For example, an inhibitor may comprise rapamycin.

UV-protectants generally comprise substances (e.g. chemical compounds, drugs) or other factors which can be used to protect cells (e.g. skin, eye, or immune cells) against damaging effects of UV radiation. Examples include active components of sunscreen compositions which may absorb UV-A and/or UV-B rays, e.g. avobenzone and octyl methoxycinnamate, and blockers of UV radiation, e.g. titanium dioxide and zinc oxide.

Anti-inflammatory agents generally comprise substances (e.g. drugs) or other factors which can be used for treatment or prevention of tissue inflammation. Examples of these substances are known in the art and include steroids (e.g. dexamethasone) or non-steroidal anti-inflammatories (NSAIDs).

Immunomodulatory agents generally comprise substances (e.g. drugs) or other factors which can be used to induce, enhance or suppress an immune response in a subject. Examples are known in the art and include steroids, methotrexate, and cyclophosphamide.

Anti-infectives generally comprise substances (e.g. chemical compounds, drugs) or other factors which are capable of treating or preventing infection. Examples are known in the art and include antibiotics, anti-viral and anti-fungal agents. For example, BPs for use in treating the skin may be used in combination with a dermatological anti-infective, e.g. topical tetracycline.

Cardiac medications for heart disease and cardiovascular conditions generally comprise substances (e.g. chemical compounds, drugs) or other factors in which can be used to treat or prevent cardiac conditions including heart disease and cardiovascular conditions. Examples include ACE inhibitors, Angiotensin II receptor blockers, digitalis medications, beta blockers, calcium channel blockers, diuretics, potassium, nitrates and anticoagulants.

As above, a BP for use in treating a given disease or condition, may be used in combination with an active agent comprising a drug or other factor for treatment or prevention of the same disease or condition. For example, a BP for use in protecting or treating skin might be used in combination with one or more dermatological treatments agents, including anti-infectives (e.g antibiotics such as topical tetracycline), steroids (e.g. hydrocortisone), or anti-scarring agents.

BPs for use in vitro may also be used in combination with one or more other active agents, for example, one or more other components of cell growth or culture media. Examples include growth factors (e.g. for stem cell expansion), and anti-oxidants.

A combination treatment of the present invention is expected to produce a synergistic or beneficial effect in treating a subject or cells for a purpose described herein. Such an effect may be determined for example by any one of the methods described herein. In the case of treatment of a specific disease or condition, this may be, for example, by one or more of the response rate, the time to disease progression, or the survival rate.

In one aspect, a synergistic or beneficial effect is achieved if the effect is superior, e.g. therapeutically or cosmetically superior, as measured by, for example, the extent of the response, the response rate, the time to disease/condition progression, side-effects experienced, or the survival period, to that achievable on applying one of the components of the combination treatment, for example, at its conventional dose or concentration. For example, the effect of a combination treatment comprising a BP compound or pharmaceutically acceptable salt or solvate thereof and a damaging cancer therapy is synergistic or beneficial if the effect is therapeutically superior to the effect achievable with the cancer therapy alone, e.g. causes fewer or less extreme side effects.

In addition, a combination treatment may be defined as affording a synergistic or beneficial effect if one of the components is applied at its conventional dose or concentration, and the other component(s) is/are applied at a reduced dose or concentration and the effect, e.g. therapeutic or cosmetic effect, as measured by, for example, the extent of the response, the response rate, the time to disease/condition progression or the survival period, is equivalent to or better than, that achievable on applying conventional amounts of the components of the combination treatment.

In particular, synergy or benefit may be deemed to be present if a conventional dose or concentration of one of the components of the combination treatment may be reduced without detriment to one or more of: the extent of the response, the response rate, the time to disease progression and survival data, in particular without detriment to the duration of the response, but with fewer and/or less troublesome side-effects than those that occur when conventional doses or concentrations of each component are used.

In one example, for a combination treatment comprising a BP compound or pharmaceutically acceptable salt or solvate thereof and a damaging cancer therapy, a synergy or benefit may be deemed to be present if a the conventional dose of the cancer therapy may be increased but with a reduction in one or more of the side-effects which would occur at that dose in the absence of the BP compound.

According to the invention, components of a combination may be administered or applied in combination or in conjunction with each other. Thus, for example, the present methods provide for administration of a BP compound or pharmaceutically acceptable salt or solvate thereof in conjunction with any one or more of the above active agents, e.g. a damaging cancer therapy.

The combination of agents may be in the form of a combined preparation of the agents, for example, a combined preparation of a BP compound or pharmaceutically acceptable salt or solvate thereof and a damaging cancer treatment agent.

The combination of agents may comprise separate formulations of one or more of the agents. For example, the combination may comprise separate formulations of a BP compound or pharmaceutically acceptable salt or solvate thereof and a damaging cancer treatment agent.

In the present methods, the agents in the combination may be administered or applied sequentially, separately and/or simultaneously. Thus for example, in a combination of a BP compound or pharmaceutically acceptable salt or solvate thereof and a damaging cancer treatment agent, the BP compound or pharmaceutically acceptable salt or solvate thereof may be administered or applied sequentially, separately and/or simultaneously with the damaging cancer treatment agent.

The skilled person will understand that where separate formulations of the agents, as defined herein, are administered sequentially or serially that this could be administration of the agents in any order. For example, where a BP compound or pharmaceutically acceptable salt or solvate thereof and a damaging cancer treatment agent are administered sequentially or serially this could be administration of a BP compound or pharmaceutically acceptable salt or solvate thereof followed by a damaging cancer treatment agent, or a damaging cancer treatment agent followed by a BP compound or pharmaceutically acceptable salt or solvate thereof.

In one embodiment the separate formulations of agents may be administered or applied in alternative dosing patterns. Where the administration of the separate formulations is sequential or separate, the delay in administering the second (or subsequent) formulation should not be such as to lose the beneficial effect (e.g. therapeutic or cosmetic effect) of the combination treatment.

Combination Products

The components of a combination treatment as described herein may be provided in a combination product.

A combination product typically comprises:

(a) a BP compound, or a pharmaceutically acceptable salt or solvate thereof; and

(b) one or more other active agents, as described herein.

The combination product is useful for protecting cells against damage, e.g. DNA damage, by a method described herein.

A combination product may comprise

(a) a BP compound, or a pharmaceutically acceptable salt or solvate thereof; and

(b) one or more other active agents as described herein;

in association with a pharmaceutically acceptable adjuvant, diluent or carrier.

The combination product provides for the administration of the components in the combination in conjunction with each other. Thus, for example, a combination product may provide for administration of a BP compound, or a pharmaceutically acceptable salt or solvate thereof in conjunction with a damaging cancer treatment agent.

A combination product, as defined herein, may be in the form of a combined preparation of the components, for example, a combined preparation of a BP compound, or a pharmaceutically acceptable salt or solvate thereof and a damaging cancer treatment agent.

A combination product, as defined herein, may comprise a kit of parts comprising separate formulations of each of the agents in the product. For example, the kit of parts may comprise separate formulations of a BP compound, or a pharmaceutically acceptable salt or solvate thereof and a damaging cancer treatment agent.

The separate formulations may be administered sequentially, separately and/or simultaneously as described herein in relation to the combination treatment methods. In one embodiment the separate formulations of the combination product, as defined herein, are administered simultaneously (optionally repeatedly). In one embodiment the separate formulations of the combination product, as defined herein, are administered sequentially (optionally repeatedly). In one embodiment the separate formulations of the combination product, as defined herein, are administered separately (optionally repeatedly).

The skilled person will understand that where the separate formulations of the combination product, as defined herein, are administered sequentially or serially that this could be administration of the agents in any order. For example, where a BP compound, or a pharmaceutically acceptable salt or solvate thereof and a damaging cancer treatment agent are administered sequentially or serially this could be administration of a BP compound, or a pharmaceutically acceptable salt or solvate thereof followed by a damaging cancer treatment agent, or a damaging cancer treatment agent followed by a BP compound, or a pharmaceutically acceptable salt or solvate thereof.

The separate formulations of the combination product, as defined herein, may be administered in alternative dosing patterns. Where the administration of the separate formulations of the combination product, as defined herein, is sequential or separate, the delay in administering the second or subsequent formulations should not be such as to lose the beneficial effect of the combination treatment.

A combination product may comprise a kit of parts comprising

(a) a BP compound, or a pharmaceutically acceptable salt or solvate thereof in association with a pharmaceutically acceptable adjuvant, diluent or carrier;

and:

(b) at least one other active agent, including any of those described herein, in association with a pharmaceutically acceptable adjuvant, diluent or carrier;

wherein the components are provided in a form which is suitable for sequential, separate and/or simultaneous administration.

The kit of parts may comprise:

a first container comprising the first of the components in the combination product, in association with a pharmaceutically acceptable adjuvant, diluent or carrier; and

a second or subsequent container comprising the second or any subsequent of the components in the combination product respectively, each component in association with a pharmaceutically acceptable adjuvant, diluent or carrier, and

a container means for containing said first and second and any subsequent containers.

The kit of parts may further comprise instructions to administer the components sequentially, separately and/or simultaneously. In one embodiment the kit of parts further comprises instructions indicating that the combination product, as defined herein, can be used for protecting against damage, e.g. DNA damage, in cells, for example in a method described herein.

A combination product, as defined herein, may comprise a pharmaceutical composition which comprises:

(a) a BP compound, or a pharmaceutically acceptable salt or solvate thereof; and

(b) at least one other active agent, including any of those described herein.

A pharmaceutical composition generally comprises a pharmaceutically acceptable adjuvant, diluent or carrier.

A combination product may comprise a pharmaceutical composition which comprises:

(a) a BP compound, or a pharmaceutically acceptable salt or solvate thereof in association with a pharmaceutically acceptable adjuvant, diluent or carrier; and

(b) at least one other active agent, including any of those described herein in association with a pharmaceutically acceptable adjuvant, diluent or carrier.

A combination product of the invention may comprise more than one BP compound or pharmaceutically acceptable salt or solvate thereof. A combination product may comprise more than one other active agent, selected from those described herein. The additional active agents may be of the same type, e.g. chemotherapeutic agents, or of different types, e.g. a chemotherapeutic agent and an additional radioprotectant.

In a combination or combination product, as defined herein, at least one agent in the combination may be linked to at least one other agent in the combination.

In one aspect therefore, the invention relates to a combination product, as defined herein, comprising

(a) a BP compound, or a pharmaceutically acceptable salt or solvate thereof; and

(b) one or more other active agents as described herein.

for use sequentially, separately and/or simultaneously in protecting cells of a subject against damage, e.g. DNA damage, for example, damage induced by radiation and/or by a chemical agent.

A combination product as described herein may be used in any of the methods described herein. Typically treatment using the combination product is in accordance with the methods of the invention described herein.

Compositions/Formulations

In one aspect, the invention relates to compositions comprising a BP compound (or pharmaceutically acceptable salt or solvate thereof) or comprising a combination product as described herein.

A composition may be for use in any of the uses or methods described herein.

For example, compositions may include: pharmaceutical compositions for therapeutic use (e.g. adjuvant compositions for use in cancer therapy, anti-inflammatory compositions, immunomodulatory compositions, cardiac medication compositions, anti-infective compositions, such as antibiotic or anti-viral compositions and wound healing compositions); skin care compositions (for therapeutic or cosmetic use, e.g. sunscreen compositions, anti-aging compositions); UV protectant compositions; cell culture media or additives for cell culture media; and animal feed compositions.

A composition may be for non-therapeutic use. For example, a composition may be for use in a cosmetic method. Examples of cosmetic products include skin care products (e.g. anti-aging products, sun-screen products).

In another example, a composition may comprise a supplement or additive for cell growth or culture media or may comprise cell growth or culture media, e.g. stem cell growth media or a supplement therefore.

A composition for use in vivo, e.g. a pharmaceutical composition or non-therapeutic composition for in vivo use, typically comprises a BP compound or pharmaceutically acceptable salt or solvate thereof, in admixture with one or more pharmaceutically acceptable excipients, carriers or diluents adjuvants, fillers, buffers, stabilisers, preservatives, lubricants, or other materials well known to those skilled in the art, and optionally one or more other active agents, such as any of those described herein.

A combined preparation of agents as described herein typically comprises the agents, as defined herein, together with one or more pharmaceutically acceptable carriers, adjuvants, excipients, diluents, fillers, buffers, stabilisers, preservatives, lubricants, or other materials well known to those skilled in the art and optionally other active agents (e.g. therapeutic agents).

Pharmaceutically acceptable excipients useful in the methods disclosed herein are conventional. Remington's Pharmaceutical Sciences, by E. W. Martin, Mack Publishing Co, Easton, Pa., 15th Edition (1975), describes compositions and formulations suitable for pharmaceutical delivery of the compounds herein disclosed.

Such formulations may further routinely contain pharmaceutically acceptable concentrations of salt, buffering agents, preservatives, antioxidants and/or compatible carriers.

Formulations may also include antioxidants and/or preservatives. As antioxidants may be mentioned tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

Formulations may be presented in unit dosage form.

Compositions for use in vitro may also comprise suitable carriers, bulking agents or other agents. For example, a composition for cell culture may comprise one or more components for growth of cells, including growth factors or anti-oxidants.

A composition which comprises a BP compound or pharmaceutically acceptable salt or solvate thereof as an active ingredient may additionally include one or more other active agents, including any of those described herein.

Delivery Routes, and Formulations

A BP compound for use as a cytoprotectant (or a composition or combination product including the BP compound) may be delivered to target cells (or to tissue, organ or organism (subject) comprising the target cells, by any suitable means.

Reference herein to administration or delivery of a BP compound to cells may include delivery to a tissue, organ or organism (subject) in which the cells are located.

Examples of administration routes and/or delivery means for delivery to a subject include: oral, parenteral, transdermal, intradermal, inter-arterial or intravenous or topical. In one example, administration may be by intravenous, inter-arterial or subcutaneous injection or infusion, or by oral administration

In the case of an animal subject, e.g. which is the source of an animal food product, a BP cytoprotectant may be included in animal feed. In the case of farmed fish, a BP compound may be included in the water containing the fish.

Where the target cells are in vitro, e.g. in cell culture, a BP cytoprotectant may, for example, be included in the cell culture media.

A composition may have a number of different forms depending on, for example, how the composition is to be applied or used. Any suitable formulation may be used. For example, formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

A composition or product may be for oral administration. In one aspect, an oral composition may comprise an oral dosage form comprising a BP compound in combination with an enhancer to improve bioavailability and/or absorption of the BP compound. For example, an enhancer may promote absorption of the BP compound at the gastrointestinal cell lining. Oral dosage forms comprising enhancers are described in, for example, U.S. Pat. No. 7,658,938 B2 and U.S. Pat. No. 8,119,159 B2.

An oral pharmaceutical formulation may be for repeated administration e.g. one a day, two a day or greater frequency. Solid dosage forms for oral administration include capsules, tablets (also called pills), powders and granules. In such solid dosage forms, the active compound is typically mixed with at least one inert, pharmaceutically acceptable excipient or carrier such as sodium citrate or dicalcium phosphate and/or one or more fillers, extenders, humectants, dissolution aids, ionic surface active agents. The active compounds may also be in micro-encapsulated form, if appropriate, with one or more excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

In one example, a composition or product may be for parenteral administration. Parenteral preparations can be administered by one or more routes, such as intravenous, subcutaneous, intradermal and infusion; a particular example is intravenous. A formulation disclosed herein may be administered using a syringe, injector, plunger for solid formulations, pump, or any other device recognized in the art for parenteral administration.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

A composition or product may be for topical administration, for example to the skin.

Formulations suitable for topical administration (e.g. transdermal, intranasal, ocular, buccal, and sublingual) may be formulated as an ointment, cream, suspension, lotion, powder, solution, paste, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Cell growth media supplements, or cell growth media, may be in any suitable form, for example, liquid, solid, paste, granules.

Amounts of Agents and Doses

Actual amounts (e.g. dosage levels) or concentrations of active ingredient (e.g. BP compound or pharmaceutically acceptable salt or solvate thereof, or other active agent) in compositions, e.g.

pharmaceutical compositions, may be varied so as to obtain an amount of active ingredient that is effective to achieve the desired effect (e.g. a protective effect as described herein), for the particular target cells and composition and (if relevant) mode of administration. Such an amount may be referred to as an effective or a protective amount. An effective (or protective) amount may, for example, be a therapeutically effective amount or a cosmetically effective amount, depending upon the purpose of use (therapeutic or cosmetic respectively).

For example, amount or dose may be varied so as to obtain an amount of active ingredient that is effective to achieve the desired protection for a therapeutic response, for a particular subject, composition, and mode of administration (referred to herein as a “therapeutically effective” amount or dose). In another example, amount or dose may be varied so as to obtain an amount of active ingredient that is effective to achieve the desired protection for a cosmetic response or benefit, for a particular subject, composition, and mode of administration (referred to herein as a “cosmetically effective” amount or dose).

The selected dosage level may, for example, depend upon the activity of the particular active ingredient, the severity of the condition being treated and the condition and, if appropriate, prior medical history of the subject being treated. However, it is within the skill of the art to start doses at levels lower than required for to achieve the desired effect and to gradually increase the dosage until the desired effect is achieved.

The dosage of a BP compound or pharmaceutically acceptable salt or solvate thereof, or of another agent in a combination described herein for a given subject or patient may be determined by an attending physician or other skilled person, taking into consideration various factors known to modify the action of drugs including severity and type of disease or condition, body weight, sex, diet, time and route of administration, other medications and other relevant factors, e.g. clinical factors. Effective dosages (e.g. therapeutically or cosmetically effective) may be determined by either in vitro or in vivo methods.

The effective amount of a BP compound or pharmaceutically acceptable salt or solvate thereof, or of another agent in a combination as described herein, to be used will depend, for example, upon the objectives, e.g. therapeutic or cosmetic objectives, the route of administration, and the condition of the subject. Accordingly, it is preferred for the therapist or other skilled person to titer the dosage and modify the route of administration as required to obtain the optimal therapeutic or other, e.g. cosmetic, effect. A typical daily dosage might range from about 0.0001 mg/kg to up to 250 mg/kg or more, depending on the factors mentioned above. Typically, the clinician or other skilled person will administer the BP compound or pharmaceutically acceptable salt or solvate thereof or combination (e.g. combination product), as described herein, until a dosage is reached that achieves the desired effect. Where separate formulations of agents in a combination are administered, the sequence in which the agents in the combination may be administered (i.e. whether and at what point sequential, separate and/or simultaneous administration takes place) may be determined by the physician or skilled person.

Administration of a combination of agents may take place as hereinbefore described, for example separate formulations of agents may be administered sequentially, separately and/or simultaneously.

“Pharmaceutically Acceptable”, Salts, Solvates, Polymorphs and Pro-Drugs

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio.

It may be convenient or desirable to prepare, purify, and/or handle a corresponding salt of BP compound or other agent described herein, for example, a pharmaceutically-acceptable salt.

A suitable pharmaceutically-acceptable salt may be, for example, an acid-addition salt which is sufficiently basic, for example an acid-addition salt with an inorganic or organic acid. Such acid-addition salts include but are not limited to, furmarate, methanesulfonate, hydrochloride, hydrobromide, citrate and maleate salts and salts formed with phosphoric and sulfuric acid. A suitable pharmaceutically-acceptable salt may be, for example, a salt which is sufficiently acidic, for example an alkali or alkaline earth metal salt. Such alkali or alkaline earth metal salts include but are not limited to, an alkali metal salt for example sodium or potassium, an alkaline earth metal salt for example calcium or magnesium, an ammonium salt, or organic amine salt for example triethylamine, ethanolamine, diethanolamine, triethanolamine, morpholine, N-methylpiperidine, N-ethylpiperidine, dibenzylamine or amino acids such as lysine.

It is also to be understood that BP (or other) compounds for use herein may exist in solvated as well as unsolvated forms such as, for example, hydrated forms. It is to be understood that the invention encompasses all such solvated forms that possess one or more BP compound property as described herein.

It is also to be understood that certain BP (or other) compounds may exhibit polymorphism, and that the invention encompasses all such forms that possess one or more BP compound property as described herein.

BP compounds may be administered in the form of a pro-drug which is broken down in the human or animal body to release a BP compound of the invention. A pro-drug may be used to alter the physical properties and/or the pharmacokinetic properties of a BP compound. A pro-drug can be formed when a BP compound contains a suitable group or substituent to which a property-modifying group can be attached.

BP pro-drugs may be particularly useful for aiding delivery of BP compounds, for example, to improved absorption, or to aid penetration of the skin in transdermal administration.

Accordingly, the present invention includes those BP compounds as defined herein when made available by organic synthesis and when made available within the human or animal body by way of cleavage of a pro-drug thereof. Accordingly, the present invention includes those BP compounds that are produced by organic synthetic means and also such compounds that are produced in the human or animal body by way of metabolism of a precursor compound, that is, a BP compound may be a synthetically-produced compound or a metabolically-produced compound.

A suitable pharmaceutically acceptable pro-drug of a BP compound is one that is based on reasonable medical judgement as being suitable for administration to the human or animal body without undesirable pharmacological activities and without undue toxicity.

Various forms of pro-drug have been described, for example in the following documents:

a) Methods in Enzymology, Vol. 42, p. 309-396, edited by K. Widder, et al. (Academic Press, 1985);

b) Design of Pro-drugs, edited by H. Bundgaard, (Elsevier, 1985);

c) A Textbook of Drug Design and Development, edited by Krogsgaard-Larsen and H. Bundgaard, Chapter 5 “Design and Application of Pro-drugs”, by H. Bundgaard p. 113-191 (1991);

d) H. Bundgaard, Advanced Drug Delivery Reviews, 8, 1-38 (1992);

e) H. Bundgaard, et al., Journal of Pharmaceutical Sciences, 77, 285 (1988);

f) N. Kakeya, et al., Chem. Pharm. Bull., 32, 692 (1984);

g) T. Higuchi and V. Stella, “Pro-Drugs as Novel Delivery Systems”, A.C.S. Symposium Series, Volume 14; and

h) E. Roche (editor), “Bioreversible Carriers in Drug Design”, Pergamon Press, 1987.

Examples of pro-drugs of BP compounds may include, for example, the bisphosphonate cyclic acetal compounds described in US 2011/0098251 A1, the contents of which, in particular the bisphosphonate cyclic acetal compounds disclosed therein, are hereby incorporated by reference.

The in vivo effects of a BP compound may be exerted in part by one or more metabolites that are formed within the human or animal body after administration of a BP compound. As stated hereinbefore, the in vivo effects of a BP compound may also be exerted by way of metabolism of a precursor compound (a pro-drug).

The description presented herein of pro-drugs of BP compounds may be applied in the same way to pro-drugs of non-BP compounds which have BP-like activity as described herein.

Treatment

Treatment (and reference to treating) as used herein includes therapeutic and/or prophylactic treatment. The term “treatment”, and the therapies encompassed by this invention, include the following and combinations thereof: (1) inhibiting, e.g. delaying initiation and/or progression of, an event, state, disorder or condition, for example arresting, reducing or delaying the development of the event, state, disorder or condition, or a relapse thereof in case of maintenance treatment or secondary prophylaxis, or of at least one clinical or subclinical symptom thereof; (2) preventing or delaying the appearance of clinical symptoms of an event, state, disorder or condition developing in an animal (e.g. human) that may be afflicted with or predisposed to the state, disorder or condition but does not yet experience or display clinical or subclinical symptoms of the state, disorder or condition; and/or (3) relieving and/or curing an event, state, disorder or condition (e.g., causing regression of the event, state, disorder or condition or at least one of its clinical or subclinical symptoms, curing a patient or putting a patient into remission).

The benefit to a subject or patient to be treated may be either statistically significant or at least perceptible to the patient or to the physician or other skilled person. It will be understood that a medicament will not necessarily produce a clinical effect in each patient to whom it is administered; thus, in any individual patient or even in a particular patient population, a treatment may fail or be successful only in part, and the meanings of the terms “treatment”, “prophylaxis” and “inhibitor” and of cognate terms are to be understood accordingly.

The term “prophylaxis” or “prophylactic treatment” includes reference to treatment therapies for the purpose of preserving health or inhibiting or delaying the initiation and/or progression of an event, state, disorder or condition, for example for the purpose of reducing the chance of, or preventing, an event, state, disorder or condition occurring. The outcome of the prophylaxis may be, for example, preservation of health or delaying the initiation and/or progression of an event, state, disorder or condition. It will be recalled that, in any individual patient or even in a particular patient population, a treatment may fail, and this paragraph is to be understood accordingly.

Treatment of a disease or condition according to the invention may be assessed by conventional means such as the response rate, the time to disease progression and/or the survival rate.

In some aspects described herein, treatment, and reference to treating, may refer to non-therapeutic treatment, e.g. cosmetic treatment. Cosmetic treatment generally does not result in a detectable clinical or therapeutic benefit.

### EXAMPLES

The invention will now be described by way of specific Examples and with reference to the accompanying Figures, which are provided for illustrative purposes only and are not to be construed as limiting upon the teachings herein.

Materials and Methods

Chemicals

Zoledronate (Zol), Alendronate, Risedronate and compounds A, B and C were dissolved in PBS. Trans, trans farnesol (FOH, Sigma, Aldrich, UK) and geranylgeraniol (GGOH, Sigma) were dissolved in ethanol at 33 mM and further diluted to a final concentration of 33 μM in MSC medium (see below for composition) for in vitro studies and E3 medium for Zebrafish experiments.

Isolation and Culture of MSC from Human Bone Marrow

Human mesenchymal stem cells (hMSC) were derived from bone marrow (BM) harvested from pelvis of patients undergoing osteotomy for reasons other than metabolic disorders at Sheffield Children's Hospital. Bone marrow was obtained following informed written parental consent in accordance with local research ethical committee approval. The BM was collected in MSC medium composed of Dulbecco's Modified Eagle's Medium (DMEM; GIBCO, Paisley, UK) and 10% Fetal Bovine Serum (FBS Hyclone, Thermo Scientific, Northumberland, UK), supplemented with 0.01% of penicillin/streptomycin (Sigma, Dorset, UK), and 0.1% heparin (Royal Hallamshire Hospital Pharmacy). Bone marrow mononuclear cells (MNC) were isolated by density gradient centrifugation at 800 g for 20 mins using Lymphocyte separation medium (1.077 g/L, PAA Laboratories, Somerset, UK). After two washes with PBS the cells plated at 8000 MNC/cm² in MSC medium and incubated at 37° C. in 5% carbon dioxide in air. After 48 hrs the non adherent cells were removed and medium was changed weekly till cells were confluent. Cultures were maintained in MSC medium and fed twice a week. When cultures reached confluence they were split using 0.5% Trypsin-1 mM EDTA (Gibco) and replated at 1000/cm². For assessment of growth kinetics the number of population doublings (PD) was calculated as Log N/Log2, where N is the number of cells at confluence divided by the number of cells at the start of the culture. When cultures were treated with Zol and/or FOH and GGOH these were administered to the cultures 72 h prior to irradiation of MSC and washed off up to 12 h afterwards depending on when the experiment was terminated.

Culture of Cell Lines

Human prostate cancer cell line PC3 cells were maintained in DMEM with Glutamax (Gibco) containing 10% FBS (Sigma), 1% penicillin (100units/ml)/streptomycin (100 μg/ml) (Sigma). Mouse prostate cancer cell line 178-2 BMA was cultured as described above with the addition of 0.1 mM 4-(2-hydroxyethyl)-1-piperazineethanesulfonic acid (Life Technologies, Gaithersburg, USA) and 0.01 mM sodium pyruvate (PAA Laboratories). Human breast cancer cell line MDA-MB-231 was cultured in RPMI 1640 with Glutamax supplemented with 10% FBS (Sigma), 0.01 mM sodium pyruvate (PAA laboratories) and penicillin (1 units/ml) and streptomycin (1 μg/ml) (Sigma). Murine myeloma cell lines 5T33 and 5TGM1 cells were cultured in RPMI 1640 media with Glutamax (Gibco) supplemented with 10% FCS (Sigma), penicillin (1units/ml) and streptomycin (1 μg/ml) (Sigma), 0.01 mM sodium pyruvate (Gibco) and 1 mM non-essential amino acids (NEAA). All cells were incubated at 37° C. in 5% CO₂.

Colony Forming Unit-Fibroblast Assay (CFU-F)

Human MSC from established cultures were plated at 10 cells/cm² in duplicates in MSC medium. The plates were incubated for 14 days at 37° C. in 5% carbon dioxide in air. At day 14 cultures were stained with Wright's Giemsa stain (VWR International, Leicestershire, UK). Briefly, media was aspirated and the plates were washed with PBS (Gibco). The plates were air dried for 5 minutes and fixed with methanol (Fisher Scientific, Loughborough, UK) for 5 minutes. Wright's Geimsa stain (VWR International) was thereafter added to the plates for 5 minutes and then plates were washed under running tap water. The purple stained colonies were counted using an inverted light microscope (Leica Microsystems, UK). Colonies containing a minimum of 50 cells were considered as one Colony forming unit-fibroblast (CFU-F).

Colony Forming Unit Assay (Cancer Cells)

Colony-forming ability of non-adherent 5TGM1 and 5T33 myeloma cancer cell lines was performed by incubating 10⁶ cells in presence or absence of Zol for 72 h and the exposed to irradiation in RPMI 1640 complete media and seeded 1% methylcellulose medium 12 h later (StemCell Technologies).

After 14 days at 37° C. 5% CO2 colonies consisting of more than 40 cells were directly scored using an inverted microscope. In the case of the other cancer lines (PC3, 178-2 BMA and MDA-MB-231) CFU assay was performed seeding cells at 35 cells/cm² in a 60 mm petri-dish and exposed to Zol for 72 h before irradiation at 3Gy. Medium was changed 12 h post-irradiation and cells were incubated at 37° C. in 5% carbon dioxide in air. At day 14 cultures were stained with Wright's Giemsa stain (VWR International) and the purple stained colonies were counted using an inverted light microscope (Leica Microsystems, UK). Colonies containing a minimum of 40 cells were considered as one colony forming unit.

Immunostaining of γH2AX for the Detection of DNA Double Stranded Breaks

DNA damage was induced by exposing hMSC to ¹³⁷Cs Gamma source. Cells were washed with PBS and fixed with 4% Para-formaldehyde for 15mins. Cells were then washed with PBS permeabilized with 0.5% Triton-X (Sigma, UK) for 2mins and blocked with 5% normal goat serum (DAKO, Glostrup, Denmark) in PBS for 1 hr. This was followed by incubation with primary antibody, anti-phospho histone H2AX (Ser139) (Millipore, Massachusetts, USA) used at 1:800 in 5% normal goat serum, overnight at 4° C. Cells were then washed in PBS and incubated in secondary Anti mouse IgG Fluorescein Isothiocyanate (FITC) conjugated (Insight Biotechnology, Santa Cruz, USA) at 1:200 in PBS for 1 hour at room temperature. The cover-slips were mounted on slides with mounting media (VectaShield) containing 4′,6-diamidino-2-phenylindole (DAPI) to stain the nuclei. Cells with double stranded breaks showed green foci in the nuclei. Cells were viewed using in an Inverted Zeiss LSM 510 NLO microscope equipped with Argon (Ar) laser (488 nm) 30 mW to image the fluorescent marker FITC and UV lamp to image DAPI stained nuclei. Five to seven fields consisting of 25 cells in total were randomly selected from a tile scan (20×) and individual cells viewed at 63× were then scored for γH2AX DNA damage foci using ImageJ 1.45 software (http://rsbweb.nih.gov/ij/).

Western Blotting

Three times 10⁶ hMSC were lysed in mammalian cell lysis buffer (Mammalian cell lysis kit, Sigma-Aldrich, Dorset, UK) containing 250 mM Tris-5 mM EDTA, 750 mM sodium chloride, 0.5% sodium dodecyl sulphate, 2.5% deoxycholic acid and 5% Igepal supplemented with 10 μl of Proteinase inhibitors cocktail (Sigma-Aldrich, Dorset, UK) containing 4-(2-aminoethyl) benzenesulfonyl fluoride (AEBSF), pepstatin A, bestatin, leupeptin, aprotinin and trans-epoxysuccinyl-L-leucyl-amido(4-guanidino)-butane (E-64) according to manufacturer instruction. Phosphatase inhibitor cocktail (Sigma) was also added when preparing lysates for the detection of AKT, pAKT, P70S6K, and p-P70S6K.

Protein lysates (50 μg) were diluted in equal volume of 2× Laemmli buffer (Gibco) containing Dithiothreitol (DTT, Sigma,UK). The samples were heated at 95° C. for 5 minutes and loaded on a 12% Tris-glycine gel. After electro-blotting, membranes were blocked with 5% Bovine serum albumin (BSA) in 0.1% Tween 20 in PBS (PBS-T) for detection of glyceraldehyde-3-phosphate dehydrogenase (GAPDH) and 5% dry milk in PBS-T for detection of unprenylated RAP1A (Santacruz Biotechnology, Santa Cruz, USA). In case of P-AKT, P-70S6K membranes were blocked with 5%BSA in 1× tris buffered saline and 0.1% tween-20 (TBS-T), where for AKT and P70S6K membranes were blocked with 5% dry milk in TBS-T. All membranes were blocked for 2 hours at room temperature. The antibody sc1482 for the detection of RAP1A (Santacruz, USA) was diluted in TBS-T at 1:1000. Antibodies for the detection of AKT, P-AKT, P-70S6K AND PP70S6K, (Cell Signalling Technologies, USA) were diluted at 1:1000. GAPDH antibody (Abcam, Cambridge) was diluted at 1:30000. Staining with primary antibodies was carried out overnight at 4° C. The secondary antibodies used were anti-mouse IgG for GAPDH (DAKO, immunoglobulin A/S, Copenhagen, Denmark) at 1:30000, anti-goat-IgG for unprenylated RAP1A (DAKO) at 1:1000 and anti-rabbit IgG (DAKO) for AKT, P-AKT, P70S6K and PP70S6K at 1:1000. The membranes were incubated for 1 hour at room temperature. Detection was carried out using enhanced chemi-luminescence plus ECL reaction kit (GE Healthcare, Buckinghamshire, UK) and quantification of protein expression was carried out using image J software.

Zebrafish Tail Amputation and Regeneration Following Irradiation

Embryos from wild-type Zebrafish (Danio Rerio H) AB strain were collected at 16 cell stage (˜1.5 hours post fertilisation (hpf)) and grown in E3 Medium at 28° C. Embryos were treated with Zoledronate (1 μM) and/or FOH, GGOH at 24 hpf. DNA damage was induced by exposure to ¹³⁷Cs Gamma source at 48 hpf and after irradiation the embryo tails were amputated and the embryos were then incubated at 28° C. for a further 12 hrs in the presence of the chemicals. At the end of this period the chemicals were washed off and the embryos were incubated in fresh medium with no chemicals. The embryos were grown up to 120 hpf, following which they were fixed in 4% PFA overnight and then mounted in 100% glycerol. The tail lengths of embryos were measured taking the anal region as starting reference point and the end of the fin fold as the ending point of measurement.

Analysis of Unprenylation in Murine Tissues Following Treatment with Zoledronate

For in vivo murine experiments C57BL6/J mice aged 8-10 weeks were used. All animals were housed in a conventional, non- specific pathogen free (SPF), mouse facility at the Medical School, University of Sheffield. Mice were fed on a commercially prepared pelleted diet and given water ad libitum. A minimum of 6 mice were used in each experimental group. Depending on the experimental groups, mice were injected either with Zoledronate(single dose, 125 μg/kg) or PBS ip, and sacrificed for tissue collection 3 days after the injection. Snap-frozen tissues were lysed for protein extraction using a tissue homogenizer and mammalian cell lysis buffer containing phosphatase and protease inhibitors. Protein lysates were run on 12% Tris-glycine gels and assessed for unprenylated RAP1A detected by sc1482 antibody RAP1A (Santacruz, USA) at 1:1000 dilution and GAPDH at 1:10000 dilution as described earlier.

Immuno-Staining of γH2AX in Murine Tissue Following Irradiation

Mice (C57BL6/J) were injected with either Zoledronate (single dose, i.p.,125 μg/kg) or PBS. On day 3 post-injection, mice received whole body irradiation (3Gy, n=6/group) using 137Cs Gamma source.

Twelve hours post irradiation mice were sacrificed for tissue collection and tissues were fixed in 4% paraformaldehyde. Tissue were placed in histological cassettes and stored in 70% ethanol prior to embedding. Paraffin wax embedded tissues were sectioned at 3-5ₗim thickness and mounted onto HEPES coated glass microscope slides. The slides were dewaxed in xylene (BDH, Leister, UK) and rehydrated by passing through a series of ethanol dilutions. Heat induced antigen retrieval in citrate buffer was carried out. The sections were then probed with primary antibody, anti-phospho histone H2AX (Ser139) (Millipore, Massachusetts, USA) at 1:800 in diluent, and incubated overnight at 4° C.

Secondary anti mouse IgG Fluorescein Isothiocyanate (FITC) conjugated (Insight Biotechnology, Santa Cruz, USA) was used at 1:200 in PBS for 1 hour incubation at room temperature. The cover-slips were mounted on slides with mounting media (VectaShield) containing 4′, 6-diamidino-2-phenylindole (DAPI) to stain the nuclei. Cells with double stranded breaks showed green foci in the nuclei. Cells were viewed using in an Inverted Zeiss LSM 510 NLO microscope equipped with Argon (Ar) laser (488 nm) 30 mW to image the fluorescent marker FITC and UV lamp to image DAPI stained nuclei. Eight to ten fields consisting of 400 cells in total were randomly selected from a tile scan (20×) and then scored for cells containing γH2AX DNA damage foci, using ImageJ 1.45 software (http://rsbweb.nih.gov/ij/). Cells were considered positive for presence of DNA damage when they showed >5 foci/cell.

Analysis of Murine Intestine Regeneration Following Irradiation and Zoledronate Treatment

Mice (C57BL6/J) were injected with either Zoledronate (single dose, i.p.,125 μg/kg) or PBS. On day 3 post-injection, Mice were exosed to whole body irradiation (9Gy; n=3/group) using 137Cs Gamma source. Within 24 hours from irradiation systemic bone marrow transplantation was performed to prevent myelotoxicity. mice were sacrificed 4 days post-irradiation for histological analysis of

Intestinal tissue. At the time of harvest this was divided with a scalpel into duodenum, ileum-jejunum, colon and rectum, and each region was further cut into 0.5 cm pieces that were placed in histological cassettes and stored in 70% ethanol prior to embedding. Tissues were paraffin embedded and sectioned at 3-5 μm thickness and mounted onto HEPES coated glass microscope slides. The slides were de-waxed in xylene (BDH, Leister, UK) and rehydrated by passing through a series of ethanol dilutions. The nuclei were stained by placing in Gill's haematoxylin (Sigma, Poole, UK), washed with water followed by staining of cytoplasm in alcoholic eosin (Sigma, Poole, UK) and washed. Slides were dehydrated through a series of graded alcohols and cleared in xylene for 3 minutes prior to mounting with DPX and coverslip to examine under microscope. Stained tissue sections were scanned on the Aperio Slide Scanner (Leica Biosystems, Newcastle, UK). To obtain villi length and crypt depth, images were analysed on the Aperio ImageScope Software (v11.2.0.780) and using the ruler tool, measurements were taken on a surface of 0.525 mm² at 3 different levels.

Statistical Analysis

All the data were analysed using Graph Pad Prism Software 5. For multiple comparisons, one way ANOVA was performed followed by Bonferroni's multiple comparison post-hoc tests. For all other comparisons Student's two-sample t-test was performed. A difference was stated to be statistically significant if the p value was <0.05 (*p<0.05; **p<0.01; ***p<0.0001).

Hydroxyapatite Binding for Bisphosphonates

Among several methods described by Ebetino et al (Bone. 2011, 49:20-33) we used hydroxyapatite column chromatography. Running buffers were prepared with potassium phosphate (1 mM) and potassium phosphate (1 M) at pH 6.8. All buffers were filtered through a disposable filter unit (0.2 μm) (Sartorius, Epsom, UK) and degassed by using an ultrasonic bath for 20 minutes before use.

The fast performance liquid chromatography (FPLC) system consisted of a Waters 650E advanced protein purification system (Millipore Corp., Waters chromatography division, Milford, Mass.), a 600E system controller and a 484 tunable absorbance detector for UV absorbance assessment. The hydroxyapatite [HAP, Ca₁₀(PO₄)₆(OH)₂] was packed in a 0.66 cm (diameter) x 6.5 cm (length) glass column (Omnifit, Bio-chem valveTM inc., Cambridge, U.K.). The column was attached to the Waters 650E system and equilibrated in the required Buffer at pH 6.8. Each compound was prepared in 1 mM potassium phosphate buffer at the corresponding pH, and 1 μmol bisphosphonate was injected into the FPLC system. BP compounds were absorbed and subsequently eluted by using a linear concentration gradient of phosphate from 1 to 1000 mM. The total run times were 24 min at a flow rate of 2 ml/min. BPs were measured by UV absorption, chemical assay, or mass spectrometric analysis. The HAP elution profile of each compound was determined in triplicate for statistical analysis (Prism, GraphPad Software, USA).

Prenyl Synthase Assay

FPP synthase activity was measured by the method of Reed and Rilling (Reed, B. C. & Rilling, H. C. (1976) Biochemistry 15, 3739-3745) with modifications. Assays were set up such that the final volume was 100 ul. The assay conditions were 50 mM Tris pH7.7, 2 mM MgCl2, 0.5 mM TCEP, 20 μg/ml BSA. For FPP synthase assays the final enzyme concentration was 10 nM. All substrates were at 10 μM final concentration each substrate, all reactions were with IPP (14C-IPP, 400Kq/μMol American Radiochem. Corp). For FPP synthase GPP was the second substrate. Bisphosphonate was added as 1/10th volume of a 10× stock solution and allowed to preincubate for 10 minutes with the enzyme in a volume of 80 ul and the reaction started by the addition of 20 ul of the combined substrate. The reaction was allowed to proceed for 4 minutes at 37° C. before being terminated by the addition of 0.2 ml of conc. HCI/Methanol (1:4) and incubated for a further 10 mins at 37° C. The reaction mixtures were then extracted with 0.4 ml of immiscible scintillation fluid (Microscint E, Perkin Elmer) to separate reaction products from unused substrate and were counted directly with a microbeta scintillation counter (Perkin Elmer).

Data were Analysed Using Graphpad Prism.

GGPP synthase assays may be carried out in the same way, but using FPP as the second substrate. Any suitable final enzyme concentration may be used, for example, 20 nM,

Experiments and Results

### Example 1

### Effect of BP (Zol) on Human Bone Marrow Derived Mesenchymal Stem Cells (hMSCs)

To test whether Zol showed an effect on extended cellular survival, human bone marrow derived mesenchymal stem cells (hMSC) were used as proof of concept. Stem cells are key players in tissue regeneration but they lose their stem cell properties with age, time in culture or following exposure to toxic agents, affecting tissue maintenance and repair.

The number of population doublings of hMSC cultured in the presence or absence of Zol (1 μM) was monitored until cells stopped growing and showed signs of senescence. Cells treated with Zol proliferated for a longer time than control cells and showed a higher clonogenic ability when replated at low density (n=3, FIG. 1A & 1B), suggesting an extended survival and a delayed loss in the quality of the stem cells, which is usually observed with time in culture.

Human MSC were exposed to osteogenic and adipogenic (differentiation supplements for 14 days and assessed for expression of osteogenic differentiation markers CBFA-1 , osteopontin(OPN), alkaline phosphatase(ALP) osteocalcin (00), and adipogenic differentiation markers Lipoprotein lipase (LPL) and peroxisome proliferator-activated receptor γ (PPAR-γ). All markers were normalised to ribosomal protein L-32. Expression of markers was increased in the presence of Zol (FIG. 1C-H).

Incidence of DNA damage foci was enumerated at passage 3(early) and p10 (late) in hMSC in presence or absence of Zol. Damage was reduced in the presence of Zol (FIG. 1I-J).

### Example 2

### Effect of BP (Zol) on DNA Damage and Clonogenic Ability of hMSCs

One of the factors affecting cellular lifespan is accumulation of DNA damage. To determine whether Zol had a protective effect on DNA damage, hMSC were exposed to 1 (FIG. 2), 3Gy and 5Gy (data not shown) irradiation in the presence or absence of Zol (1 μM) and DNA damage was evaluated as the number of DNA double strand breaks by counting the number of γH2AX foci. Zol produced a significant reduction in γH2AX DNA damage foci at 4, 12 and 24 hours after irradiation (2A-B, n=3) and this was mirrored by a complete rescue of the clonogenic ability of the cells (FIG. 2C, n=3).

### Example 3

### Mechanism of Action of BP (Zol)

Zol is known to exert its anti-osteoclast action by inhibiting the farnesyl pyrophosphate synthase

(FPPS) enzyme in the mevalonate pathway and thereby reducing prenylation of small GTPases, such as Rap, Rac, Rho, Rheb (FIG. 3A). It was therefore important to determine whether the effect on DNA damage repair was mediated by the same mechanism of inhibition of the mevalonate pathway or by a different mechanism. To this end, levels of unprenylated Rap1A were measured in hMSC exposed to increasing amounts of Zol. A dose response study showed that increasing dosing with Zol led to increased DNA repair, with an increased expression of unprenylated Rap1a (FIG. 3B) and reduced number of DNA damage foci (FIG. 3C, n=3). Moreover when prenylation was restored by re-introduction of the intermediates geranylgeraniol (GGOH) and farnesol (FOH), downstream of FPP synthase, the effect on DNA damage was reversed (FIG. 3D, n=3), suggesting that Zol promoted DNA repair by inhibiting the mevalonate pathway.

To further test whether this effect was mediated by inhibition of the mevalonate pathway two isomeric BPs with high (compound A) and low (compound B) inhibitory potency on the FPP synthase enzyme were used. hMSC were exposed to 1Gy irradiation in presence or absence of compounds A and B and γH2AX DNA damage foci were measured after 4 h). Only when hMSC were irradiated in presence of compound A which had high affinity for FPP synthase a significant reduction in DNA damage was seen further confirming that the action of Zol on DNA repair is mediated by (or at least partially dependent upon) inhibition of the mevalonate pathway (FIG. 4, n=3).

### Example 4

### Effect of BP (Zol) on Tissue Regeneration

To determine whether Zol properties also resulted in protection of tissue regeneration, zebrafish embryo caudal fin was used. Due to its accessibility, its fast and robust regeneration and its simple architecture, the zebrafish caudal fin is an established model of regeneration of a relatively complex tissue that is easy to amputate, is not required for viability, and completely regenerates in a short time frame (7-10 days). Following amputation, proliferation of blastema cells, mesenchymal-like cells with stem cell properties, and concomitant patterning and differentiation, results in the regeneration of the amputated portions of the damaged tissue. When Zebrafish is exposed to irradiation this process is significantly reduced but is rescued by the addition of Zol (FIG. 5, n=15). Interestingly, similar to what seen in hMSC the repair process is abrogated by the addition of FOH and GGOH which reverse the inhibition of the mevalonate pathway (FIG. 6, n=15)

### Example 5

### Effect of BP (Zol) on DNA Damage in Murine Multiple Myeloma Cell Line 5TGM1

Zol is used as therapy in bone disease of cancers including multiple myeloma, osteosarcoma, and breast cancer. To determine whether Zol showed a similar mechanism of DNA repair in cancer cells potentially limiting the action of cytotoxic therapies, a murine multiple myeloma line, 5TGM1, was irradiated in presence and absence of Zol and examined for γH2AX DNA damage foci 4 h after irradiation. No significant difference was found in the number of foci when cells were exposed to irradiation in the presence of Zol (FIG. 7, n=3)

### Example 6

### Mechanism of Action of BP (Zol)

The inventors hypothesized that the above-identified action of Zol on DNA damage in cells might be mediated by inhibition of mTOR. Inhibition of mTOR signalling is implicated in lifespan extension in model organisms and its dysregulation has been associated with. Moreover downstream effectors of mTOR such as FOXO3A have been recognised as important effectors for the recruitment of DNA damage response genes such as ATM. Moreover, this signalling pathway contains prenylated proteins such as RAS and Rheb which could be modulated by Zol.

To test this, the inventors assessed the phosphorylation state of two effectors downstream of TORC1 and TORC2, p-P70S6K and pAKT (Ser473) respectively (FIG. 7A), in hMSC in presence or absence of Zol. To determine whether mTOR signalling was differentially affected by Zol in cancer cells, changes in the osteosarcoma line MG63 were also examined. The line 5TGM1 was not used as it showed very low level of pAKT expression. A significant decrease in pAKT and p-P70S6K was observed in hMSC when they were exposed to Zol for 72 h (n=3, FIGS. 8B and C). In contrast no significant difference was found when the same molecules were measured in MG63 (n=3) suggesting that Zol may have a differential action in normal compared with cancer cells (FIGS. 8B and C)

### Example 7

### Effect of BP Having Lower Bone Affinity (Compound C) on DNA Damage in hMSCs

In the present protocol, Zol was washed off soon after the administration of the damaging agent to avoid potential interference of Zol with the cell cycle as this would be expected with inhibition of mTOR signalling. If Zol was to be given to promote protection of normal tissues it may be advantageous that it is in a form that has low affinity to bone but equal potency. To test whether other BPs with low affinity to bone are as effective, Compound C, whose affinity for the bone mineral, hydroxyapatite, is less than that of Zol, was used. Compared to Zol, hMSC exposed to Compound C showed equal ability to repair DNA double strand breaks compared with Zol at the same concentration of 1 μM (FIG. 9), suggesting that a similar effect will be obtained with other nitrogen-containing BPs. The ability of potent BPs with lower affinity for bone mineral to exert these effects may enable them to be more effective in vivo, since less drug will be bound to bone, leaving more drug free to act on other cells.

### Example 8

### Determining HAP Affinity and FPPS Inhibition of a Number of Bisphosphonate Compounds.

Affinity for hydroxyapatite (HAP) and inhibition of FPPS was determined for a number of bisphosphonate compounds, using the methods described in the Materials and methods section. Results are shown in FIG. 10.

1. A bisphosphonate (BP) compound, or a pharmaceutically acceptable salt or solvate or pro-drug thereof, for use in a subject as a cytoprotectant for protecting non-cancerous cells against radiation-induced damage and/or damage induced by a chemical agent.

2. A bisphosphonate (BP) compound, or a pharmaceutically acceptable salt or solvate or pro-drug thereof, for use according to claim 1 wherein the damage comprises DNA damage.

3. A bisphosphonate (BP) compound, or a pharmaceutically acceptable salt or solvate or pro-drug thereof, for use according to claim 1 or 2 wherein the damage comprises cell death, premature cell aging, aberrant cell function, aberrant cell division ,increased risk of developing primary cancer and/or damage induced by reactive-oxygen species (ROS).

4. A bisphosphonate (BP) compound, or a pharmaceutically acceptable salt or solvate or pro-drug thereof, for use according to any of claims 1 to 3 wherein the non-cancerous cells comprise adult stem cells.

5. A bisphosphonate (BP) compound, or a pharmaceutically acceptable salt or solvate or pro-drug thereof, for use according to any of claims 1 to 4 wherein the radiation comprises radiotherapy.

6. A bisphosphonate (BP) compound, or a pharmaceutically acceptable salt or solvate thereof, for use according to any of claims 1 to 5 wherein the chemical agent comprises chemotherapy.

7. A bisphosphonate (BP) compound, or a pharmaceutically acceptable salt or solvate or pro-drug thereof, for use according to any of the preceding claims wherein the subject is a cancer patient and wherein the cytoprotectant protects non-cancerous cells of the subject from damage induced by cancer radiotherapy and/or cancer chemotherapy.

8. A bisphosphonate (BP) compound, or a pharmaceutically acceptable salt or solvate or pro-drug thereof, for use as a cytoprotective adjuvant in cancer chemotherapy and/or cancer radiotherapy in a subject.

9. A bisphosphonate (BP) compound, or a pharmaceutically acceptable salt or solvate or pro-drug thereof, for use according to claim 7 or 8 wherein one or more side effects of the cancer radiotherapy and/or chemotherapy is reduced.

10. A bisphosphonate compound or a pharmaceutically acceptable salt or solvate or pro-drug thereof, for use according to any of claims 1 to 4 wherein the radiation comprises solar radiation.

11. A bisphosphonate (BP) compound, or a pharmaceutically acceptable salt or solvat or pro-drug e thereof, for use according to claim 10 wherein the non-cancerous cells comprise skin cells.

12. A bisphosphonate (BP) compound, or a pharmaceutically acceptable salt or solvate or pro-drug thereof, for use in protecting a subject against damage by solar radiation.

13. A bisphosphonate (BP) compound, or a pharmaceutically acceptable salt or solvate or pro-drug thereof, for use according to any of the preceding claims wherein the BP compound protects the subject against the development of a first or a second primary cancer.

14. A bisphosphonate compound or a pharmaceutically acceptable salt or solvate or pro-drug thereof, for use according to any of claims 1 to 4, wherein the damage is associated with a disease or condition selected from: physical or chemical tissue trauma, radiation-induced tissue trauma, ischaemia or a condition associated with ischaemia, aging or an age-related disorder, an inflammatory disorder, a degenerative disease or disorder, a stem cell disease or disorder, chronic obstructive pulmonary disease, cardiac failure, infection and an autoimmune disorder.

15. A bisphosphonate (BP) compound, or a pharmaceutically acceptable salt or solvate or pro-drug thereof, for use according to claim 14 wherein:
(a) physical or chemical tissue trauma is selected from wounding, cancer chemotherapy, thermal damage, water damage, or damage due to exposure of cells to naturally occurring or synthetic chemicals;
(b) radiation-induced tissue trauma is selected from damage due to cancer radiotherapy, solar radiation, UV radiation, infrared, X-rays or gamma-rays;
(c) ischaemia is selected from ischaemic heart disease, ischaemia of the bowel, ischaemia of the brain or ischaemia of limb tissue;
(d) a condition associated with ischaemia is selected from: atherosclerosis, ischaemic heart disease, heart failure, tachycardia, hypoglycaemia, hypotension, thromboembolism, sickle cell disease, frostbite, peripheral artery occlusive disease, blood vessel rupture or anaemia;
(e) an inflammatory disorder is selected from inflammatory bowel disease (IBD), colitis, bursitis, cystitis, dermatitis, phlebitis, rhinitis, tendonitis, tonsillitis, vasculitis, acne, asthma, autoimmune diseases, chronic prostatitis, glomerulonephritis, hypersensitivities, pelvic inflammatory disease, reperfusion injury, sarcoidosis, transplant rejection or inflammatory myopathies;
(f) a degenerative disease or disorder comprises Alzheimer's disease; and/or
(g) a stem cell disease or disorder comprises Fanconi anaemia.
(h) infection is selected from bacterial infection, viral infection, or fungal infection; and
(i) an autoimmune disorder is selected from Addison's disease, coeliac disease, dermatomyositis, Graves disease, Hashimoto's thyroiditis, multiple sclerosis, myasthenia gravis, pernicious anaemia, reactive arthritis, rheumatoid arthritis, Sjogren syndrome or systemic lupus erythematosus (SLE).

16. A bisphosphonate (BP) compound, or a pharmaceutically acceptable salt or solvate or pro-drug thereof, for use according to any of claims 1 to 9 wherein the subject is a stem cell donor and/or a stem cell recipient in stem cell transplantation or gene therapy.

17. Use of a bisphosphonate (BP) compound, or a pharmaceutically acceptable salt or solvate or pro-drug thereof, for the manufacture of a cytoprotectant medicament for protecting non-cancerous cells of a subject against radiation-induced damage and/or damage induced by a chemical agent.

18. A method of protecting non-cancerous cells against radiation-induced damage and/or damage induced by a chemical agent, the method comprising administering an effective amount of a bisphosphonate (BP) compound, or a pharmaceutically acceptable salt or solvate or pro-drug thereof, to the cells.

19. Use of a bisphosphonate (BP) compound, or a pharmaceutically acceptable salt or solvate or pro-drug thereof as a cytoprotectant for protecting non-cancerous cells against radiation-induced damage and/or damage induced by a chemical agent.

20. A method or use according to any of claims 17 to 19 wherein the bisphosphonate (BP) compound, or pharmaceutically acceptable salt or solvate or pro-drug thereof, is administered to the cells in vitro.

21. A use or a method according to any of claims 17 to 20 wherein the damage comprises DNA damage.

22. Use of a bisphosphonate (BP) compound, or pharmaceutically acceptable salt or solvate or pro-drug thereof for preparing induced pluripotent stem cells.

23. A method of preparing induced pluripotent stem cells, the method comprising:
administering an effective amount of a bisphosphonate (BP) compound, or a pharmaceutically acceptable salt or solvate or pro-drug thereof, to one or more multipotent cells; and
preparing induced pluripotent stem cells from the multipotent cells.

24. Use of a bisphosphonate (BP) compound, or pharmaceutically acceptable salt or solvate or pro-drug thereof as a cytoprotectant for treating cosmetic signs of aging in a subject or for protecting a subject against cosmetic damage by solar radiation.

25. A bisphosphonate (BP) compound, or a pharmaceutically acceptable salt or solvate thereof or pro-drug for use as a UV protectant.

26. Use of a bisphosphonate (BP) compound, or a pharmaceutically acceptable salt or solvate or pro-drug thereof for reducing one or more visible signs of aging in skin.

27. A bisphosphonate (BP) compound, or a pharmaceutically acceptable salt or solvate or pro-drug thereof, for use, or a method or use according to any of the preceding claims wherein the bisphosphonate (BP) compound, or a pharmaceutically acceptable salt or solvate thereof is administered in combination with one or more agents selected from: cancer radiotherapy; cancer chemotherapeutic agents; cytoprotective agents; inhibitors of the mevalonate pathway; inhibitors of mTOR signalling; anti-inflammatory agents; immunomodulatory agents; UV-protectants;anti-infectives, and cardiac medications for heart disease and cardiovascular conditions.

28. A combination product for use in protecting non-cancerous cells of a subject against radiation-induced damage and/or damage induced by a chemical agent, the combination product comprising a bisphosphonate (BP) compound or a pharmaceutically acceptable salt or solvate or pro-drug thereof, and one or more agents selected from: cancer radiotherapy; cancer chemotherapeutic agents; cytoprotective agents; inhibitors of the mevalonate pathway; inhibitors of mTOR signalling; anti-inflammatory agents; immunomodulatory agents; UV-protectants;anti-infectives, and cardiac medications for heart disease and cardiovascular conditions.

29. A combination product for use according to claim 25 wherein the combination product comprises:
(a) a bisphosphonate (BP) compound, or a pharmaceutically acceptable salt or solvate or pro-drug thereof, in association with a pharmaceutically acceptable adjuvant,diluent or carrier; and
(b) at least one agent selected from cancer radiotherapy; cancer chemotherapeutic agents; cytoprotective agents; inhibitors of the mevalonate pathway; inhibitors of mTOR signalling; anti-inflammatory agents; immunomodulatory agents; UV-protectants; anti-infectives, and cardiac medications for heart disease and cardiovascular conditions; wherein the at least one agent is in association with a pharmaceutically acceptable adjuvant,diluent or carrier;
wherein the components are provided in a form which is suitable for sequential, separate and/or simultaneous administration.

30. A cytoprotective adjuvant composition comprising a bisphosphonate (BP) compound, or a pharmaceutically acceptable salt or solvate or pro-drug thereof, and a suitable carrier, excipient or diluent.

31. A UV protectant composition or sunscreen composition comprising a bisphosphonate (BP) compound, or a pharmaceutically acceptable salt or solvate or pro-drug thereof, and a suitable carrier, excipient or diluent.

32. A skincare composition, comprising a bisphosphonate (BP) compound, or a pharmaceutically acceptable salt or solvate or pro-drug thereof, and a suitable carrier, excipient or diluent.

33. Cell growth media composition, or an additive composition for cell culture media comprising a bisphosphonate (BP) compound, or a pharmaceutically acceptable salt or solvate or pro-drug thereof, and a suitable carrier, excipient or diluent.

34. A bisphosphonate (BP) compound or a pharmaceutically acceptable salt or solvate or pro-drug thereof for use, or a method, or a use, or a combination product for use, or a composition, according to any of the preceding claims wherein the BP compound comprises a nitrogen-containing bisphosphonate (N-BP) compound.

35. A bisphosphonate (BP) compound or a pharmaceutically acceptable salt or solvate or pro-drug thereof for use, or a method, or a use, or a combination product for use, or a composition, according to any of the preceding claims wherein the BP compound comprises any one or more of Zoledronate, Compound A, Compound B or Compound C.

36. A bisphosphonate (BP) compound or a pharmaceutically acceptable salt or solvate or pro-drug thereof for use in protecting a subject against radiation-induced damage and/or damage induced by a chemical agent.

37. A bisphosphonate (BP) compound or a pharmaceutically acceptable salt or solvate or pro-drug thereof for use in reducing one or more side effect of radiotherapy and/or chemotherapy in a subject.

38. A compound selected from:
(a) a phosphono-phosphinate compound; and
(b) an inhibitor of FPPS enzyme;
or a pharmaceutically acceptable salt or solvate or pro-drug of (a) or (b), for use as a cytoprotectant for protecting non-cancerous cells of a subject against radiation-induced damage and/or damage induced by a chemical agent.

39. A method of protecting non-cancerous cells against radiation-induced damage and/or damage induced by a chemical agent, the method comprising administering an effective amount of
a) a phosphono-phosphinate compound; or
(b) an inhibitor of FPPS enzyme; or
a pharmaceutically acceptable salt or solvate or pro-drug of (a) or (b) to the cells.

40. A compound or a method according to claim 38 or 39 wherein the phosphono-phosphinate compound comprises a pyridylaminomethane phosphonoalklyphosphinate compound.
